(19) United States
(12) Patent Application Publication
(10) Pub. No.: US 2017/0204123 A1
(43) Pub. Date: Jul. 20, 2017

(54) Gold (I)-Phosphine Compounds as Anti-Bacterial Agents

(71) Applicant: Auspherix Limited, Stevenage, Hertfordshire (GB)

(72) Inventors: Ian Holmes, Stevenage, Hertfordshire (GB); Alan Naylor, Royston, Hertfordshire (GB); Gabriel Negoita-Giras, Cambridge, Cambridgeshire (GB); Jonathan Powell, Cambridge, Cambridgeshire (GB); Ian Charles, Norwich, Norfolk (GB); Albert Dagmar, Stevenage, Hertfordshire (GB)

(21) Appl. No.: 15/314,478

(22) PCT Filed: May 28, 2015
(86) PCT No.: PCT/GB2015/051550

§ 371 Date: Nov. 28, 2016

(30) Foreign Application Priority Data
May 28, 2014 (GB) 1409401.5
Feb. 6, 2015 (GB) 1501969.8

(51) Int. Cl. C07F 9/6561; A61K 31/665; A61K 45/06; C07F 9/59; A61K 31/66; C07F 9/6512; C07F 9/6509; C07F 9/6506; C07F 9/28; C07F 9/655; A61K 31/675

(52) U.S. Cl. CPC C07F 9/6561; C07F 9/6552; A61K 31/665; A61K 45/06; C07F 9/592; A61K 31/675; C07F 9/65122; C07F 9/650917; C07F 9/65068; C07F 9/28; A61K 31/66

(57) ABSTRACT

A compound of formula (I) for use in the prevention or treatment of a bacterial infection wherein R^{P1} is either methyl, ethyl, isopropyl, cyclohexyl or phenyl; R^{P2} is selected from methyl, ethyl, isopropyl, cyclohexyl and phenyl; R^{P3} is either ethyl, isopropyl, cyclohexyl, phenyl or pyridyl; A is either S or Se; R^{A} is selected from wherein each of Y¹, Y², Y³, Y⁴ and Y⁹ is independently selected from CH or N, wherein at least three of Y¹, Y², Y³, Y⁴ and Y⁹ are CH; V is selected from O, CH—OR^{O1}, N—CO₂—R^{C2} or N—R^{N2}; one of V, Y⁶, Y⁷ and Y⁸ is selected from CH and N, and the others are CH; X is selected from NH, S or O; R^{C1} is selected from O—R^{O2} or NHR^{N1}; R^{O1} is selected from H and C₁₋₃ unbranched alkyl; R^{O2} is C₁₋₃ unbranched alkyl; R^{N1} is selected from H and C₁₋₃ unbranched alkyl; R^{N2} is C₁₋₃ unbranched alkyl; R^{C2} is either C₁₋₃ unbranched alkyl or C₃₋₄ branched alkyl; R^{C3} is selected from C₁₋₃ unbranched alkyl and C₂H₄CO₂H; R^{C4} is either H or Me; R^{C5} is either H or Me; R^{C6} represents one or two optional methyl substituents; and n is an integer from 2 to 8.

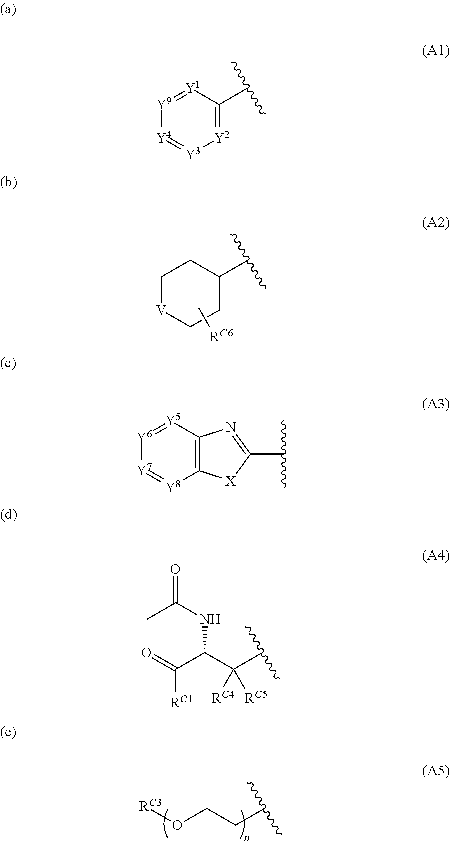

The present invention relates to gold (I)-phosphine compounds, and their use as inhibitors of growth of Gram-positive and/or Gram-negative bacteria. The present invention also relates to using such compounds for the prevention and/or treatment of bacterial infection.

The global rise of bacteria and other microorganisms resistant to antibiotics and antimicrobials in general, poses a major threat. Deployment of massive quantities of antimicrobial agents into the human ecosphere during the past 60 years has introduced a powerful selective pressure for the emergence and spread of antimicrobial-resistant bacterial pathogens. The World Health Organization has highlighted antimicrobial resistance (AMR) as an issue of global concern in 2014. AMR is now present in all parts of the world with the incidence of antibiotic resistance (ABR) in bacteria that cause common infections (e.g. pneumonia, bloodstream infections and urinary tract infections) rendering many historically efficacious antibiotics ineffective. Of particular concern are hospital-acquired infections caused by highly resistant bacteria such as the ESKAPE pathogens (Enterococcus faecium, Staphylococcus aureus, Klebsiella pneumoniae, Acinetobacter baumannii, Pseudomonas aeruginosa, and Enterobacter species), Escherichia coli, Coagulase-negative staphylococci and Clostridium difficile. Additionally, failure of last resort third-generation cephalosporins for the treatment of gonorrhea has now been reported in 10 countries raising the possibility that gonorrhea may soon become untreatable in the absence of new antibacterial agents.

The biological activity of gold(I) and gold(III) complexes has been studied historically and salts of both have been demonstrated to possess antimicrobial activity against a range of pathogens (Gli\check{s}ić, B. D. & Djuran M. I., Dalton Trans., 2014, 43, 5950-5969).

Gold(I) is a soft Lewis acid and preferentially complexes with soft donor atoms such as sulfur, selenium and phosphorous. Examples of such complexes used clinically include gold thiomalate, aurothioglucose and auranofin:

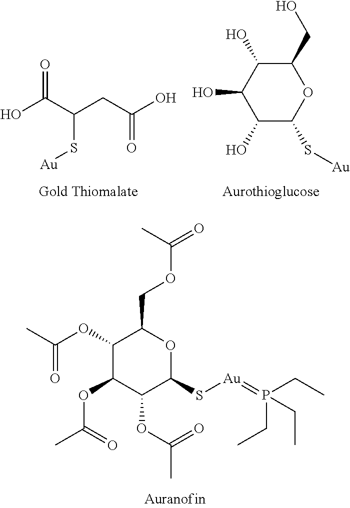

Auranofin, a second generation orally bioavailable gold(I) based treatment for rheumatoid arthritis (RA), has been identified as inhibiting the in vitro growth of S. aureus (Oxford strain) with an MIC of 0.6-0.9 μg/mL and V. cholerae with an MIC of 2.5 μg/mL. These observations reinforce multiple literature reports of the antimicrobial activity of auranofin and other gold(I) compounds against a range of bacterial pathogens (Madeira, J M., Inflammopharmacology, 2012, 20, 297-306; Jackson-Rosario, S, J. Biol. Inorg. Chem., 2009, 14(4), 507-519; Novelli, F., Farmaco, 1999, 54, 232-236; Shaw, C F, Chem Rev., 1999, 99(9), 2589-2600; Rhodes, M D, J. Inorg. Biochem., 1992, 46, 129-142 and Fricker, S P, Transition Met. Chem., 1996, 21, 377-383).

A first aspect of the present invention provides a compound of formula (I):

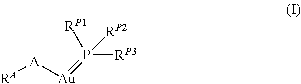

for use in the prevention or treatment of a bacterial infection wherein:

R^{P1} is either methyl, ethyl, isopropyl, cyclohexyl or phenyl;

R^{P2} is selected from methyl, ethyl, isopropyl, cyclohexyl and phenyl;

R^{P3} is either ethyl, isopropyl, cyclohexyl, phenyl or pyridyl;

A is either S or Se;

R^{A} is selected from:

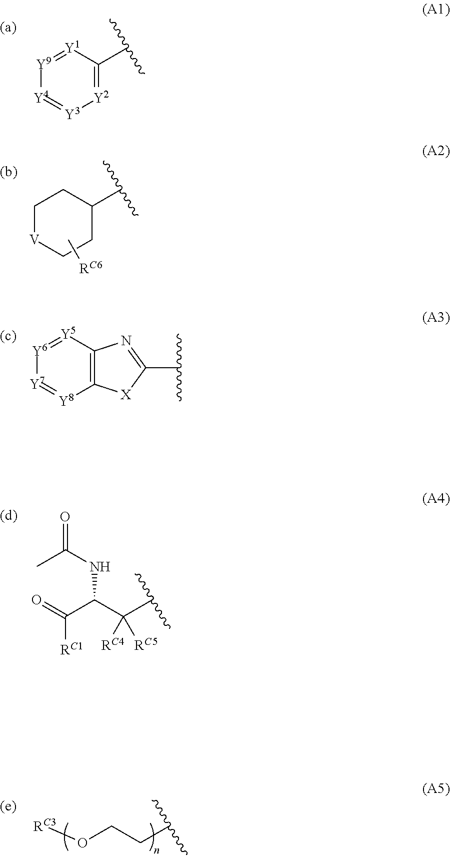

wherein:
each of Y¹, Y², Y³, Y⁴ and Y⁹ is independently selected from CH or N, wherein at least three of Y¹, Y², Y³, Y⁴ and Y⁹ are CH;
V is selected from O, CH—OR^{O1}, N—CO₂—R^{C2} or N—R^{N2};
one of Y⁵, Y⁶, Y⁷ and Y⁸ is selected from CH and N, and the others are CH;
X is selected from NH, S or O;
R^{C1} is selected from O—R^{O2} or NHR^{N1};
R^{O1} is selected from H and C₁₋₃ unbranched alkyl;
R^{O2} is C₁₋₃ unbranched alkyl;
R^{N1} is selected from H and C₁₋₃ unbranched alkyl;

R^{N2} is C₁₋₃ unbranched alkyl; R^{C2} is either C₁₋₃ unbranched alkyl or C₃₋₄ branched alkyl;
R^{C3} is selected from C₁₋₃ unbranched alkyl and C₂H₄CO₂H;
R^{C4} is either H or Me;
R^{C5} is either H or Me;
R^{C6} represents one or two optional methyl substituents; and
n is an integer from 2 to 8.

The first aspect of the invention also provides the use of a compound of formula (I) in the manufacture of a medicament for the treatment and/or prevention of a bacterial infection. The first aspect of the invention further provides the treatment of a human or animal patient afflicted with a bacterial infection, comprising administering to said patient an effective amount of a pharmaceutical composition containing a compound of formula (I).

In the first aspect, the bacterial infection prevented and/or treated may be infection by one or more Gram-positive bacteria. The bacterial infection prevented and/or treated may be infection by one or more Gram-negative bacteria.

A second aspect of the present invention provides a compound of formula (I):

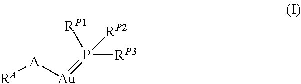

wherein:

R^{P1} is either methyl, ethyl, isopropyl, cyclohexyl or phenyl;

R^{P2} is selected from methyl, ethyl, isopropyl, cyclohexyl and phenyl;

R^{P3} is either ethyl, isopropyl, cyclohexyl, phenyl or pyridyl;

A is either S or Se;

R^{A} is selected from:

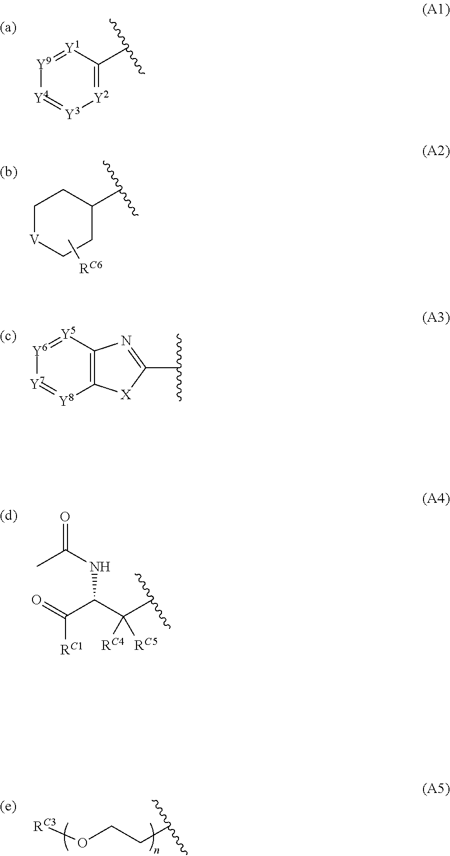

wherein:
each of Y¹, Y², Y³, Y⁴ and Y⁹ is independently selected from CH or N, wherein at least three of Y¹, Y², Y³, Y⁴ and Y⁹ are CH;
V is selected from O, CH—OR^{O1}, N—CO₂—R^{C2} or N—R^{N2};
one of Y⁵, Y⁶, Y⁷ and Y⁸ is selected from CH and N, and the others are CH;
X is selected from NH, S or O;
R^{C1} is selected from O—R^{O2} or NHR^{N11};
R^{O1} is selected from H and unbranched C₁₋₃ alkyl;
R^{O2} is C₁₋₃ unbranched alkyl;
R^{N1} is selected from H and C₁₋₃ unbranched alkyl;
R^{N2} is C₁₋₃ unbranched alkyl;
R^{C2} is either C₁₋₃ unbranched alkyl or C₃₋₄ branched alkyl;
R^{C3} is selected from C₁₋₃ unbranched alkyl and C₂H₄CO₂H;
R^{C4} is either H or Me;
R^{C5} is either H or Me;
R^{C6} represents one or two optional methyl substituents; and
n is an integer from 2 to 8;
with the proviso that the compound is not:

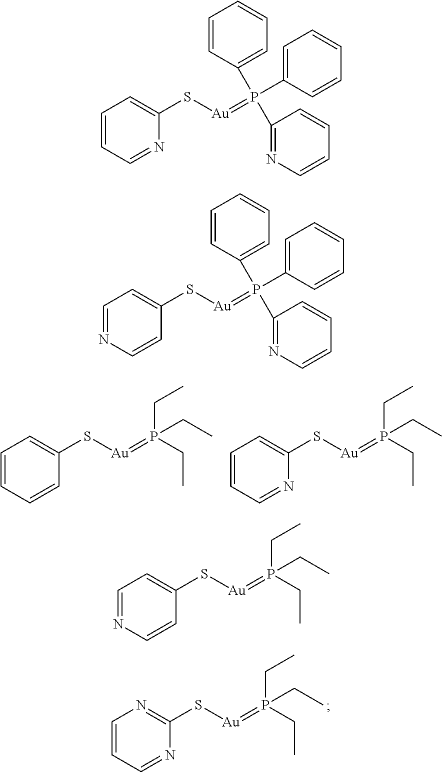

and
with the further proviso that when A is S, and R^{A} is A3, then one of Y⁵, Y⁶, Y⁷ and Y⁸ is N.

In some embodiments of the second aspect, if A is S, R^{A} is (A1), then Y¹, Y² and Y⁹ are CH, and Y³ and Y⁴ are N.

A third aspect of the present invention provides a pharmaceutical composition comprising a compound of the second aspect of the invention. The pharmaceutical composition may also comprise a pharmaceutically acceptable diluent or excipient. The third aspect of the present invention also provides the use of a compound of the second aspect of the invention in a method of therapy.

Further aspects of the invention relate generally to the use of the compounds of the present invention to inhibit microbial growth, sensitize the inhibition of microbial growth, inhibit biofilm formation or development, disrupt existing biofilms, reduce the biomass of a biofilm, and sensitize a biofilm and microorganisms within the biofilm to an antimicrobial agent.

In one aspect the invention relates to a method for inhibiting biofilm formation, comprising exposing a biofilm-forming microorganism to an effective amount of a compound of the invention. In some embodiments a compound of the invention is coated, impregnated or otherwise contacted with a surface or interface susceptible to biofilm formation. In some embodiments, the surface is a surface of a medical device such as: medical or surgical equipment, an implantable medical device or prosthesis (for example, venous catheters, drainage catheters (e.g. urinary catheters), stents, pacemakers, contact lenses, hearing-aids, percutaneous glucose sensors, dialysis equipment, drug-pump related delivery cannula, prostheses such as artificial joints, implants such as breast implants, heart valves, medical fixation devices such as rods, screws, pins, plates, or devices for wound repair such as sutures, and wound dressings such as bandages). In particular embodiments, the biofilm or biofilm-forming microorganism is on a bodily surface of a subject and exposure of the biofilm or biofilm-forming microorganism to a compound of the invention is by administration of the compound of the invention to the subject. In such instances, the biofilm or biofilm-forming microorganism may be associated with an infection, disease or disorder suffered by the subject or to which the subject is susceptible. In a related aspect of the invention, a medical device (such as those exemplified above) coated or impregnated with a compound of the invention is provided.

In another aspect the invention relates to a method for reducing the biomass of a biofilm and/or promoting the dispersal of microorganisms from a biofilm, comprising exposing the biofilm to an effective amount of a compound of the invention.

In yet another aspect the invention relates to a method for dispersing or removing, removing, or eliminating a biofilm, comprising exposing the biofilm to an effective amount of a compound of the invention. In some embodiments the biofilm is an existing, preformed or established biofilm.

In a further aspect the invention relates to a method for killing microorganisms within a biofilm, comprising exposing the biofilm to an effective amount of a compound of the invention. In some embodiments the biofilm is an existing, preformed or established biofilm.

In a yet further aspect the invention relates to a method of sensitizing a microorganism in a biofilm to an antimicrobial agent by exposing the biofilm to an effective amount of a compound of the invention. In some embodiments the antimicrobial agent is an antibiotic (e.g. rifampicin, gentamicin, erythromycin, lincomycin, linezolid or vancomycin) or an antifungal agent.

In one aspect the invention relates to a compound of the invention for use in a method of dispersing, removing or eliminating an existing biofilm, inhibiting biofilm formation, reducing the biomass of a biofilm, promoting the dispersal of microorganisms from a biofilm, killing microorganisms within a biofilm, sensitizing a microorganism in a biofilm to an antimicrobial agent, treating or preventing an infection, disease or disorder caused by a biofilm, inhibiting the growth of a microbial persister cell, killing a microbial persister cell, or treating or preventing an infection, disease or disorder caused by or associated with a microbial persister cell.

In another aspect the invention relates to a compound of the invention for use in a method of treating or preventing an infection, disease or disorder treatable by dispersing, removing or eliminating an existing biofilm, inhibiting biofilm formation, reducing the biomass of a biofilm, promoting the dispersal of microorganisms from a biofilm, killing microorganisms within a biofilm, sensitizing a microorganism in a biofilm to an antimicrobial agent, inhibiting the growth of a microbial persister cell, killing a microbial persister cell, or treating or preventing an infection, disease or disorder caused by or associated with a microbial persister cell.

In some aspects, the biofilm comprises bacteria, such as, for example, multi-drug resistant bacteria. In some aspects the bacteria are Gram positive bacteria. In some aspects the bacteria are Gram negative bacteria. In particular examples, the biofilm comprises, consists essentially of, or consists of S. aureus. In some aspects, the S. aureus is methicillin-resistant S. aureus (MRSA). In some embodiments, the biofilm comprises, consists essentially of, or consists of A. baumannii. In other embodiments, the biofilm comprises, consists essentially of, or consists of K. pneumoniae. In other embodiments, the biofilm comprises, consists essentially of, or consists of one or more of the bacteria listed in Table 1 herein. In further embodiments, the biofilms comprise bacterial species, including but not limited to, Staphylococcus spp., Streptococcus spp., Enterococcus spp., Listeria spp. and Clostridium spp., Klebsiella spp., Acinetobacter spp., Pseudomonas spp., Burkholderia spp., Erwinia spp., Haemophilus spp., Neisseria spp., Escherichia spp, Enterobacter spp., Vibrio spp. and/or Actinobacillus spp.

In some aspects, biofilm comprises lower eukaryotes, such as yeast, fungi, and filamentous fungi, including, but not limited to Candida spp., Pneumocystis spp., Coccidioides spp., Aspergillus spp., Zygomycetes spp., Blastoschizomyces spp., Saccharomyces spp., Malassezia spp., Trichosporon spp. and Cryptococcus spp. Example species include C. albicans, C. glabrata, C. parapsilosis, C. dubliniensis, C. krusei, C. tropicalis, A. fumigatus, and C. neoforms.

The biofilm may comprise one species of microorganism, or comprise two or more species of microorganism, i.e. be a mixed species biofilm. The mixed species biofilms may include two or more species of bacteria, two or more species of lower eukaryote (e.g. two or more fungal species, such as unicellular fungi, filamentous fungi and/or yeast), and/or both bacteria and lower eukaryotes, such as one or more species of bacteria and one or more species of lower eukaryotes. For example, the methods, uses and compositions provided herein are applicable to biofilms comprising one or more species of bacteria and one or more species of fungi, such as a yeast, unicellular fungi and/or filamentous fungi. The mixed species biofilm may thus comprise 2, 3, 4, 5, 10, 15, 20 or more species of microorganism, and the microorganisms within the biofilm may be bacteria and/or lower eukaryotes, such as unicellular fungi, filamentous fungi and/or yeast.

In one aspect the invention relates to a method for killing persister cells or inhibiting the growth of a microbial persister cell, comprising exposing the persister cell to an effective amount of a compound of the invention

In another aspect the invention relates to a method for reducing the number, density or proportion of persister cells in a microbial population, comprising exposing the persister cell to an effective amount of a compound of the invention. In some embodiments the number, density or proportion of persister cells in a microbial population is reduced by at least 10% compared to an otherwise identical population not exposed to a compound of the invention; for example, at least 20%, at least 30%, at least 40%, at least 50%, at least 60%, at least 70%, at least 80%, at least 90%, at least 95%, at least 98%, at least 99%, at least 99.9%, or at least 99.99%.

In a further aspect the invention relates to a method of preventing the formation of microbial persister cells in a microbial population, the method comprising exposing the population to an effective amount of a compound of the invention.

In some aspects the persister cell is a bacterial or fungal persister cell. In some examples, the persister cell is a Gram negative bacterium. In some examples, the persister cell is a Gram positive bacterium. In some examples, the persister cell is a small colony variant. In particular embodiments, the persister cells are Staphylococcus spp. (including Staphylococcal SCVs), such as S. aureus (including methicillin resistant S. aureus (MRSA)), S. epidermidis, and S. capitis. In further embodiments, the persister cells are Pseudomonas spp. such as P. aeruginosa; Burkholderia spp. such as B. cepacia and B. pseudomallei; Salmonella serovars, including Salmonella Typhi; Vibrio spp. such as V. cholerae; Shigella spp.; Brucella spp. such as B. melitensis; Escherichia spp. such as E. coli; Lactobacillus spp. such as L. acidophilus; Serratia spp. such as S. marcescens; Neisseria spp. such as N. gonorrhoeae, or Candida spp., such as C. albicans.

The compounds of the invention can act together with other antimicrobial agents, allowing for increased efficacy of anti-microbial action. Accordingly, for any aspect described herein comprising exposing a biofilm, biofilm-forming microorganism, or a microbial persister cell to a compound of the invention, the present invention provides a corresponding further aspect comprising exposing the biofilm or biofilm-forming microorganism to a combination of compounds of the invention and at least one additional antimicrobial agent, such as, for example, an antibiotic or an anti-fungal agent. In particular examples, the antibiotic is selected from rifampicin, gentamicin, erythromycin, lincomycin and vancomycin.

The methods described herein may be performed, for example, in vivo, ex vivo, or in vitro.

### Definitions

C₁₋₃ unbranched alkyl: The term “C₁₋₃ unbrached alkyl” as used herein, pertains to a monovalent moiety obtained by removing a hydrogen atom from a C₁₋₃ unbranched saturated hydrocarbon compound having from 1 to 3 carbon atoms. Thus, the term comprises the groups methyl, ethyl and n-propyl.

C₃₋₄ branched alkyl: The term “03-4 branched alkyl” as used herein, pertains to a monovalent moiety obtained by removing a hydrogen atom from a C₃₋₄ branched saturated hydrocarbon compound having from 3 to 4 carbon atoms. Thus, the term comprises the groups iso-propyl, iso-butyl, sec-butyl and tert-butyl.

Microbe/Microorganism: The terms “microbe/microorganism” as used herein pertain to bacteria and lower eukaryotes, such as fungi, including yeasts, unicellular fungi and filamentous fungi.

Antimicrobial agent: The term “antimicrobial agent” as used herein pertains to any agent that, alone or in combination with another agent, is capable of killing or inhibiting the growth of one or more species of microorganism. Antimicrobial agents include, but are not limited to, antibiotics, antifungals, detergents, surfactants, agents that induce oxidative stress, bacteriocins and antimicrobial enzymes (e.g. lipases, proteinases, pronases and lyases) and various other proteolytic enzymes and nucleases, peptides and phage. Reference to an antimicrobial agent includes reference to both natural and synthetic antimicrobial agents. Examples of antimicrobial agents include fluoroquinolones, aminoglycosides, glycopeptides, lincosamides, cephalosporins and related beta-lactams, macrolides, nitroimidazoles, penicillins, polymyxins, tetracyclines, and any combination thereof. For example, the methods of the present invention can employ acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; chlorhexidine, cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin G benzathine, penicillin G potassium, penicillin G procaine, penicillin G sodium, penicillin V, penicillin V benzathine, penicillin V hydrabamine, and penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; zorbamycin; bifonazolem; butoconazole; clotrimazole; econazole; fenticonazole; isoconazole; ketoconazole; miconazolel omoconazolel oxiconazolel sertaconazolel sulconazolel tioconazolel; albaconazole; fluconazole; isavuconazole; itraconazole; posaconazole; ravuconazole; terconazole; voriconazole; abafungin; amorolfin; butenafine; naftifine; terbinafine; anidulafungin; caspofungin; and micafungin.

Biofilm: The term “biofilm” as used herein pertains to any three-dimensional, matrix-encased microbial community displaying multicellular characteristics. Accordingly, the term biofilm includes surface-associated biofilms as well as biofilms in suspension, such as flocs and granules. Biofilms may comprise a single microbial species or may be mixed species complexes, and may include bacteria as well as fungi, algae, protozoa, or other microorganisms.

Reducing the biomass of a biofilm: The term “reducing the biomass of a biofilm” is used herein to mean reducing the biomass of an area of a biofilm exposed to an effective amount of a compound of the invention as compared to the biofilm biomass of the area immediately before exposure to a compound of the invention. In some embodiments the “biomass” is the mass of cells present in the area of biofilm in addition to the extracellular polymeric substance (EPS) of the biofilm matrix. In some embodiments the “biomass” is only the mass of cells present in the area of biofilm (that is, the mass of the EPS is not counted as “biomass”). In some embodiments the biomass of the area of a biofilm exposed to an effective amount of a compound of the invention is at least 10% less than the biofilm biomass of the area immediately before exposure to a compound of the invention, the mass of the otherwise identical area of a biofilm which has not been exposed to a compound of the invention, for example, at least 20%, at least 30%, at least 40%, at least 50%, at least 60%, at least 70%, at least 80%, at least 90%, at least 95%, at least 98%, or at least 99% less than the biofilm biomass of the area immediately before exposure to a compound of the invention. In some embodiments the area of biofilm compared is 10⁻⁶ m²; in other embodiments the area of biofilm compared is 10⁻⁵ m², 10⁻⁴ m², or 10⁻³ m². In some embodiments a biofilm whose biomass has been reduced by at least 95% is deemed to have been “eliminated”, “dispersed” or “removed”. In some embodiments a biofilm whose biomass has been reduced by at least 99% is deemed to have been “eliminated”, “dispersed” or “removed”. In some embodiments a biofilm whose biomass has been reduced by at least 99.9% is deemed to have been “eliminated”, “dispersed” or “removed”. In some embodiments the change in biofilm biomass is assessed by a method comprising the steps of: i) washing the area of biofilm to remove non-adherent (planktonic) microorganisms, ii) assessing the area of biofilm biomass (i.e. the biomass “immediately before exposure to a compound of the invention”), iii) exposing the area of biofilm (or an otherwise identical area) to an effective amount of a compound of the invention for a period of time (for example, 24 hours), iv) washing the biofilm to remove non-adherent (planktonic) microorganisms, and v) assessing the area of biofilm biomass to obtain the ‘post-exposure’ biomass.

Promoting the dispersal of microorganisms from a biofilm: The term “promoting the dispersal of microorganisms from a biofilm” is used herein to mean reducing the number of microorganisms present in an area of a biofilm exposed to an effective amount of a compound of the invention as compared to the number of microorganisms present in the area immediately before exposure to a compound of the invention. In some embodiments the number of microorganisms in the area of a biofilm exposed to an effective amount of a compound of the invention is at least 10% less than the number of microorganisms present in the area immediately before exposure to a compound of the invention, for example, at least 20%, at least 30%, at least 40%, at least 50%, at least 60%, at least 70%, at least 80%, at least 90%, at least 95%, at least 98%, or at least 99% less than the number of microorganisms present in the area immediately before exposure to a compound of the invention. In some embodiments the change in number of microorganisms in an area of biofilm is assessed by a method comprising the steps of: i) washing the biofilm to remove non-adherent (planktonic) microorganisms, ii) counting the remaining microorganisms to obtain a ‘pre-exposure’ microorganism count (i.e. the count “immediately before exposure to a compound of the invention”), iii) exposing the biofilm to an effective amount of a compound of the invention for a period of time (for example, 24 hours), iv) washing the biofilm to remove non-adherent (planktonic) microorganisms, and v) counting the remaining microorganisms to obtain the ‘post-exposure’ microorganism count. In some embodiments a biofilm where number of microorganisms in an area has been reduced by at least 95% is deemed to have been “eliminated”, “dispersed” or “removed”. In some embodiments a biofilm where number of microorganisms in an area has been reduced by at least 99% is deemed to have been “eliminated”, “dispersed” or “removed”. In some embodiments a biofilm where number of microorganisms in an area has been reduced by at least 99.9% is deemed to have been “eliminated”, “dispersed” or “removed”.

Killing microorganisms within a biofilm: The term “killing microorganisms within a biofilm” is used herein to mean reducing the number of live microorganisms present in an area of a biofilm exposed to an effective amount of a compound of the invention as compared to the number of live microorganisms present in the area immediately before exposure to a compound of the invention. In some embodiments the biofilm is an existing, preformed or established biofilm. In some embodiments the number of live microorganisms in the area of a biofilm exposed to an effective amount of a compound of the invention is at least 10% less than the number of live microorganisms present in the area immediately before exposure to a compound of the invention, for example, at least 20%, at least 30%, at least 40%, at least 50%, at least 60%, at least 70%, at least 80%, at least 90%, at least 95%, at least 98%, or at least 99% less than the number of live microorganisms present in the area immediately before exposure to a compound of the invention. In some embodiments the change in number of microorganisms in an area of biofilm is assessed by a method comprising the steps of: i) washing the area biofilm to remove non-adherent (planktonic) microorganisms, ii) manually disperse the biofilm into solution (using, for example, scraping, sonication, and vortexing), iii) prepare serial dilutions, plat, and culture to estimate the number of colony forming unit (cfu) in the area of biofilm, iv) provide an otherwise identical area of biofilm and expose it to an effective amount of a compound of the invention for a period of time (for example, 24 hours), v) manually disperse the biofilm and estimate cfu as described above to obtain the ‘post-exposure’ microorganism count.

Dispersal: The term “dispersal” as used herein pertains to any to a biofilm and microorganisms making up a biofilm means the process of detachment and separation of cells and a return to a planktonic phenotype or behaviour of the dispersing cells.

Exposing: The term “exposing” as used herein means generally bringing into contact with. Exposure of a biofilm or biofilm-forming microorganism to an agent (e.g. a compound of the invention) includes administration of the agent to a subject harbouring the microorganism or biofilm, or otherwise bringing the microorganism or biofilm into contact with the agent itself, such as by contacting a surface on which the biofilm or biofilm-forming microorganism are present with the agent. In some embodiments, the biofilm or biofilm-forming microorganisms are exposed to a compound of the invention by coating, impregnating or otherwise contacting a surface or interface susceptible to biofilm formation to an effective amount of the compound. Surfaces that may be exposed, coated, or impregnated with a compound of the invention include those present in a range of industrial and domestic settings, including but not limited to, domestic, medical or industrial settings (e.g. medical and surgical devices, and surfaces within hospitals, processing plants and manufacturing plants), as well as internal and external surfaces of the body of a subject. In the present disclosure the terms “exposing”, “administering” and “contacting” and variations thereof may, in some contexts, be used interchangeably.

Inhibiting: The term “inhibiting” and variations thereof such as “inhibition” and “inhibits” as used herein in relation to microbial growth refers to any microbiocidal or microbiostatic activity of an agent (e.g. a compound of the invention) or composition. Such inhibition may be in magnitude and/or be temporal or spatial in nature. Inhibition of the growth of a microorganism by an agent can be assessed by measuring growth of the microorganism in the presence and absence of the agent. The growth can be inhibited by the agent by at least or about 10%, 15%, 20%, 25%, 30%, 35%, 40%, 45%, 50%, 55%, 60%, 65%, 70%, 75%, 80%, 85%, 90%, 95% or more compared to the growth of the same microorganism that is not exposed to the agent.

The term “inhibiting” and variations thereof such as “inhibition” and “inhibits” as used herein in relation to biofilms means complete or partial inhibition of biofilm formation and/or development and also includes within its scope the reversal of biofilm development or processes associated with biofilm formation and/or development. Further, inhibition may be permanent or temporary. The inhibition may be to an extent (in magnitude and/or spatially), and/or for a time, sufficient to produce the desired effect. Inhibition may be prevention, retardation, reduction or otherwise hindrance of biofilm formation or development. Such inhibition may be in magnitude and/or be temporal or spatial in nature. Inhibition of the formation or development of a biofilm by a compound of the invention can be assessed by measuring biofilm mass or microbial growth in the presence and absence of a compound of the invention. The formation or development of a biofilm can be inhibited by a compound of the invention by at least about 10%, 15%, 20%, 25%, 30%, 35%, 40%, 45%, 50%, 55%, 60%, 65%, 70%, 75%, 80%, 85%, 90%, 95% or more compared to the formation or development of a biofilm that is not exposed to a compound of the invention.

Sensitize: The terms “sensitize” or “sensitizing” as used herein mean making a biofilm or microorganisms within a biofilm more susceptible to an antimicrobial agent. The sensitizing effect of a compound of the invention, on a biofilm or microorganisms within the biofilm can be measured as the difference in the susceptibility of the biofilm or microorganisms (as measured by, for example, microbial growth or biomass of the biofilm) to a second antimicrobial agent with and without administration of the compound. The sensitivity of a sensitized biofilm or microorganism (i.e. for example, a biofilm or microorganism exposed to an agent such as a compound of the invention) to a antimicrobial agent can be increased by at least about 10%, 20%, 30%, 40%, 50%, 60%, 70%, 80%, 90%, 100%, 150%, 200%, 250%, 300%, 350%, 400%, 450%, 500% or more compared to the sensitivity of an unsensitized biofilm or microorganism (i.e. a biofilm or microorganism not exposed to the agent). In some embodiments sensitizing effect of a compound of the invention on a biofilm or microorganisms within the biofilm can be measured by the difference in Minimum Inhibitory Concentration (MIC) of a second antimicrobial administered either in combination with a compound of the invention, or alone. For example, in some embodiments the MIC of a combination of a compound of the invention and the second antimicrobial is at least 10% lower than the MIC of the second antimicrobial administered alone; such as at least 20% lower, at least 30% lower, at least 40% lower, at least 50% lower, at least 60% lower, at least 70% lower, at least 80% lower, at least 90% lower, at least 95% lower, at least 99% lower, or at least 99.9% lower than the MIC of the second antimicrobial administered alone. The sensitization of a microorganism may also occur outside of a biolfim.

Surface: The term “surface” as used herein includes both biological surfaces and non-biological surfaces. Biological surfaces typically include surfaces both internal (such as organs, tissues, cells, bones and membranes) and external (such as skin, hair, epidermal appendages, seeds, plant foliage) to an organism. Biological surfaces also include other natural surfaces such as wood or fibre. A non-biological surface may be any artificial surface of any composition that supports the establishment and development of a biofilm.

Such surfaces may be present in industrial plants and equipment, and include medical and surgical equipment and medical devices, both implantable and non-implantable. Further, for the purposes of the present disclosure, a surface may be porous (such as a membrane) or non-porous, and may be rigid or flexible.

Infection, disease or disorder caused by a biofilm/Infection, disease or disorder caused by or associated with a microbial persister cell: The term “Infection, disease or disorder caused by a biofilm” as used herein is used to describe conditions, diseases and disorders associated with, characterised by, or caused by biofilms and biofilm-forming microorganisms. Similarly, The term “Infection, disease or disorder caused by or associated with a microbial persister cell” as used herein is used to describe conditions, diseases and disorders associated with, characterised by, or caused by microbial persister cells. For example, a variety of microbial infections are known to be associated with biofilm formation and/or persister cells, such as cellulitis, impetigo, mastitis, otitis media, bacterial endocarditis, sepsis, toxic shock syndrome, urinary tract infections, pulmonary infections (including pulmonary infection in patients with cystic fibrosis), pneumonia, dental plaque, dental caries, periodontitis, bacterial prostatitis and infections associated with surgical procedures or burns. For example, S. aureus and S. epidermidis cause or are associated with cellulitis, impetigo, mastitis, otitis media, bacterial endocarditis, sepsis, toxic shock syndrome, urinary tract infections, pulmonary infections (including pulmonary infection in patients with cystic fibrosis), pneumonia, dental plaque, dental caries and infections associated with surgical procedures or burns. In other examples, K. pneumoniae can cause or be associated with pneumonia, sepsis, community-acquired pyogenic liver abscess (PLA), urinary tract infection, and infections associated with surgical procedures or burns. In further examples, A. baumannii can cause or be associated with bacteremia, pneumonia, meningitis, urinary tract infection, and. and infections associated with wounds. In still further examples, P. aeruginosa can cause or be associated with respiratory tract infections (including pneumonia), skin infections, urinary tract infections, bacteremia, infection of the ear (including otitis media, otitis externa and otitis interna), endocarditis and bone and joint infections such as osteomyelitis. Candida spp. such as C. albicans, Cryptococcus spp. such as C. neoformans, as well as other fungi such as Trichosporon spp., Malassezia spp., Blastoschizomyces spp., Coccidioides spp. and Saccharomyces spp. (e.g. S. cerevisiae) may cause or be associated with infections related to the implantation or use of medical or surgical devices, such as catheterization or implantation of heart valves.

Persister cell(s): The term “persister cell(s)” as used herein pertains to metabolic variants of wild type microbial cells that are phenotypically characterized by their slow growth rate, which is typically 30%, 25%, 20%, 15%, 10%, 5% or less of the growth rate of the wild-type counterpart. In some embodiments, the persister cells are dormant and have, for example, no detectable cell division in a 24 hour period. Further, persister cells typically form colonies that are approximately 30%, 25%, 20%, 15%, 10%, 5% or less of the size of the colonies formed by their wild-type counterparts. Reference to persister cells includes reference to persister cells of any microbial genera or species, including, but not limited to, bacterial and lower eukaryotic, such as fungal, including yeast, persister cells. In some examples, the persister cell is a Gram negative bacterium. In some examples, the persister cell is a Gram positive bacterium. Exemplary persister cells include, but are not limited to, those of Staphylococcus spp., such as S. aureus, S. epidermidis, and S. capitis; Pseudomonas spp. such as P. aeruginosa; Burkholderia spp. such as B. cepacia and B. pseudomallei; Salmonella serovars, including Salmonella Typhi; Vibrio spp. such as V. cholerae; Shigella spp.; Brucella spp. such as B. melitensis; Escherichia spp. such as E. coli; Lactobacillus spp. such as L. acidophilus; Serratia spp. such as S. marcescens; Neisseria spp. such as N. gonorrhoeae, as well as Candida spp., such as C. albicans.

### FURTHER EMBODIMENTS

R^{P1-3}

In some embodiments, R^{P1} is methyl. In other embodiments, R^{P1} is ethyl. In other embodiments, R^{P1} is isopropyl. In other embodiments, R^{P1} is phenyl.

In some embodiments, R^{P2} is methyl. In other embodiments, R^{P2} is ethyl. In other embodiments, R^{P2} is isopropyl. In other embodiments, R^{P2} is phenyl.

In some embodiments, R^{P3} is ethyl. In other embodiments, R^{P3} is isopropyl. In other embodiments, R^{P3} is phenyl. In other embodiments, R^{P3} is pyridyl.

In some embodiments, R^{P1} and R^{P3} and the same. In other embodiments, R^{P1} and R^{P2} are the same.

In some embodiments, R^{P1}, R^{P2} and R^{P3} are ethyl. In other embodiments, R^{P1}, R^{P2} and R^{P3} are isopropyl.

In some embodiments, R^{P1} and R^{P3} are phenyl and R^{P2} is methyl.

In some embodiments, R^{P1} and R^{P2} are methyl and R^{P3} is phenyl.

In some embodiments, R^{P1}, R^{P2} and R^{P3} are cyclohexyl.

A

In some embodiments, A is S.

In some embodiments, A is Se.

RA

In some embodiments, R^{A} is A1:

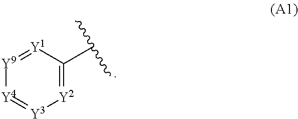

In some embodiments, one of Y¹, Y², Y³, Y⁴ and Y⁹ is N. In some of these embodiments, Y¹ is N and Y², Y³, Y⁴ and Y⁹ are CH. In others of these embodiments, Y³ is N and Y¹, Y², Y⁴ and Y⁹ are CH. In others of these embodiments, Y⁴ is N and Y¹, Y², Y³ and Y⁹ are CH. In these embodiments, A1 is pyridyl.

In some embodiments, two of Y¹, Y², Y³, Y⁴ and Y⁹ are N. In some of these embodiments, Y¹, Y⁴ and Y⁹ are CH and Y² and Y³ are N. In others of these embodiments, Y², Y⁴ and Y⁹ are CH and Y¹ and Y³ are N. In others of these embodiments, Y³, Y⁴ and Y⁹ are CH and Y¹ and Y² are N. In some of these embodiments, Y¹ and Y⁴ are N and Y², Y³ and Y⁹ are CH. In others of these embodiments, Y² and Y⁴ is N and Y¹, Y³, and Y⁹ are CH. In others of these embodiments, Y³ and Y⁴ are N and Y¹, Y² and Y⁹ are CH. In others of these embodiments, Y³ and Y⁹ are N and Y¹, Y² and Y⁴ are CH. In these embodiments, A1 is selected from pyrimidinyl, pyridazinyl and pyrazinyl.

In some embodiments, all of Y¹, Y², Y³, Y⁴ and Y⁹ are CH, i.e. A1 is phenyl.

In some embodiments, RA is A2:

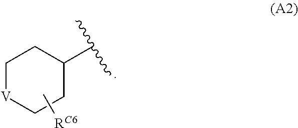

In some of these embodiments, V is O.

In other of these embodiments, V is CH—OR^{O1}, where R^{O1} is selected from H and C₁₋₃ unbranched alkyl. In some of these embodiments, R^{O1} is H. In others of these embodiments, R^{O1} is C₁₋₃ unbranched alkyl, e.g. methyl, ethyl, n-propyl.

In other of these embodiments, V is N—CO₂—R^{C2}, where R^{C2} is either C₁₋₃ unbranched alkyl or C₃₋₄ branched alkyl. In some of these embodiments, R^{C2} is C₁₋₃ unbranched alkyl, i.e.

methyl, ethyl, n-propyl. In others of these embodiments, R^{C2} is C₃₋₄ branched alkyl, i.e. iso-propyl, iso-butyl, sec-butyl and tert-butyl.

In other of these embodiments, V is N—R^{N2}, where R^{N2} is C₁₋₃ unbranched alkyl, i.e. methyl, ethyl, n-propyl. In some embodiments, R^{N2} is methyl.

In some of these embodiment, there are no optional methyl substituents (represented by R^{C6}).

In other of these embodiments, there is a single methyl substituent represented by R^{C6}. In other of these embodiments, there are two methyl substituents represented by R^{C6}.

In some embodiments, RA is A3:

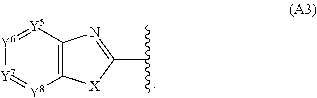

In some of these embodiments, X is NH. In others of these embodiments, X is O.

In some of these embodiments, all of Y⁵, Y⁶, Y⁷ and Y⁸ are CH. In others of these embodiments, one of Y⁵, Y⁶, Y⁷ and Y⁸ is N. In some of these embodiments, Y⁵ may be N. In some of these embodiments Y⁶ may be N. In some of these embodiments Y⁷ may be N. In some of these embodiments Y⁸ may be N.

In some embodiments, RA is A4:

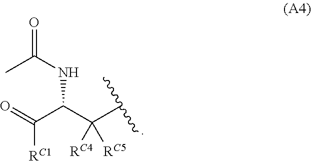

In some of these embodiments, R^{C1} is O—R^{O2}. R^{O2} is C₁₋₃ unbranched alkyl, i.e. methyl, ethyl, n-propyl.

In others of these embodiments, R^{C1} is NHR^{N1}. In some of these embodiments, R^{N1} is H. In others of these embodiments, R^{N1} is C₁₋₃ unbranched alkyl, i.e. methyl, ethyl, n-propyl.

In some of these embodiments, R^{C4} and R^{C5} are both H.

In other of these embodiments, R^{C4} is H and R^{C5} is Me.

In other of these embodiments, R^{C4} and R^{C5} are both Me.

In some embodiments, R^{A} is A5:

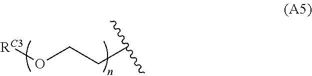

In some of these embodiments, R^{C3} is C₁₋₃ unbranched alkyl, i.e. methyl, ethyl, n-propyl.

In others of these embodiments R^{C3} is C₂H₄CO₂H.

In some of these embodiments n is an integer from 4 to 8. In some of these embodiments, n is 7 or 8.

In some embodiments of the present invention, the compound is of formula (Ia):

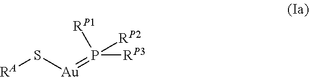

wherein:

R^{P1} is either methyl, ethyl, isopropyl or phenyl;

R^{P2} is selected from methyl, ethyl, isopropyl and phenyl;

R^{P3} is either ethyl, isopropyl or phenyl;

R^{A} is selected from:

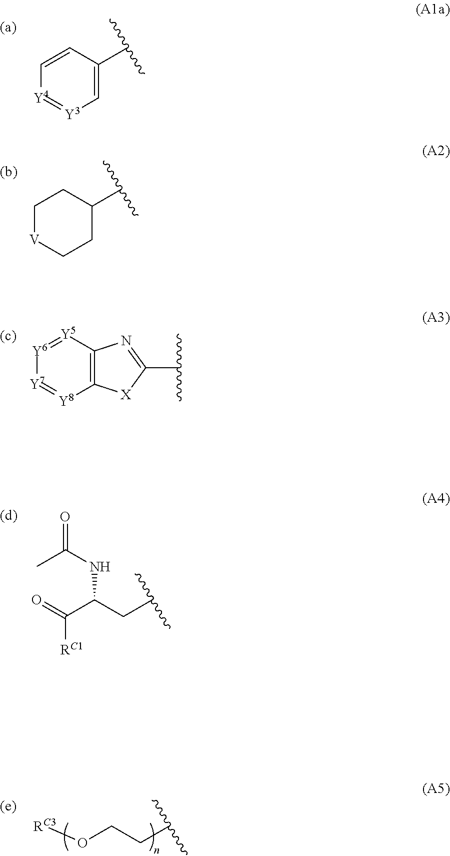

wherein:
Y³ and Y⁴ are independently selected from N and CH, and at least one is N;
V is selected from O, CH—OR^{O1} or N—CO₂—R^{C2};
one of Y⁵, Y⁶, Y⁷ and Y⁸ is N, and the others are CH;
X is selected from NH or O;

R^{C1} is selected from O—R^{O2} or NHR^{N11};
R^{O1} is selected from H and unbranched C₁₋₃ alkyl;
R^{O2} is C₁₋₃ unbranched alkyl;
R^{N1} is selected from H and C₁₋₃ unbranched alkyl;
R^{C2} is either C₁₋₃ unbranched alkyl or C₃₋₄ branched alkyl;
R^{C3} is selected from C₁₋₃ unbranched alkyl and C₂H₄CO₂H; and
n is an integer from 2 to 8.

In some embodiments of the present invention, the compound is of formula (Ib):

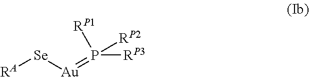

wherein:

R^{P1} is either methyl, ethyl, isopropyl or phenyl;

R^{P2} is selected from methyl, ethyl, isopropyl and phenyl;

R^{P3} is either ethyl, isopropyl or phenyl;

R^{A} is selected from:

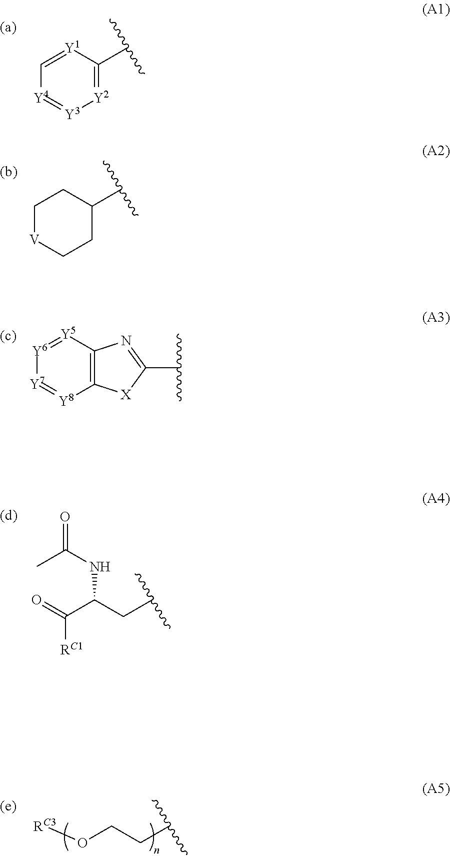

wherein:
each of Y¹, Y², Y³ and Y⁴ is independently selected from CH or N, wherein at least one of Y¹, Y², Y³ and Y⁴ is N and at least two of Y¹, Y², Y³ and Y⁴ is CH;
V is selected from O, CH—OR^{O1} or N—CO₂—R^{C2};
one of Y⁵, Y⁶, Y⁷ and Y⁸ is selected from CH and N, and the others are CH;
X is selected from NH or O;
R^{C1} is selected from O—R^{O2} or NHR^{N11};
R^{O1} is selected from H and unbranched C₁₋₃ alkyl;
R^{O2} is C₁₋₃ unbranched alkyl;
R^{N1} is selected from H and C₁₋₃ unbranched alkyl;
R^{C2} is either C₁₋₃ unbranched alkyl or C₃₋₄ branched alkyl;
R^{C3} is selected from C₁₋₃ unbranched alkyl and C₂H₄CO₂H; and
n is an integer from 2 to 8.

Particular embodiments of the invention are shown in the examples.

Bacterial Infections

Bacteria that cause infection of humans include, but are not limited to, those set out below in Table 1.

TABLE 1

| Genus | Important species | Gram negative/positive |
|---|---|---|
| Bordetella | Bordetella pertussis | Gram-negative |
| Borrelia | Borrelia burgdorferi | Gram-negative |
| Brucella | Brucella abortus | Gram-negative |
| | Brucella canis | |
| | Brucella melitensis | |
| | Brucella suis | |
| Burkholderia | Burkholderia cepacia | Gram-negative |
| Campylobacter | Campylobacter jejuni | Gram-negative |
| Chlamydia and | Chlamydia pneumoniae | (not |
| Chlamydophila | Chlamydia trachomatis | Gram-stained) |
| | Chlamydophila psittaci | |
| Clostridium | Clostridium botulinum | Gram-positive |
| | Clostridium difficile | |
| | Clostridium perfringens | |
| | Clostridium tetani | |
| Corynebacterium | Corynebacterium diphtheriae | Gram-positive |
| Enterobacter | Enterobacter cloacae | Gram-negative |
| Enterococcus | Enterococcus faecalis | Gram-positive |
| | Enterococcus faecium | |
| Escherichia | Escherichia coli | Gram-negative |
| Francisella | Francisella tularensis | Gram-negative |
| Haemophilus | Haemophilus influenzae | Gram-negative |
| Helicobacter | Helicobacter pylori | Gram-negative |
| Klebsiella | Klebsiella oxytoca | Gram-negative |
| | Klebsiella pneumoniae | |
| Legionella | Legionella pneumophila | Gram-negative |
| Leptospira | Leptospira interrogans | Gram-negative |
| Listeria | Listeria monocytogenes | Gram-positive |
| Moraxella | Moraxella catarrhalis | Gram-negative |
| Neisseria | Neisseria gonorrhoeae | Gram-negative |
| | Neisseria meningitidis | |
| Proteus | Proteus vulgaris | Gram-negative |
| Pseudomonas | Pseudomonas aeruginosa | Gram-negative |
| Rickettsia | Rickettsia rickettsii | Gram-negative |
| Salmonella | Salmonella typhi | Gram-negative |
| | Salmonella typhimurium | |
| Shigella | Shigella sonnei | Gram-negative |
| Staphylococcus | Staphylococcus aureus | Gram-positive |
| | Staphylococcus epidermidis | |
| | Staphylococcus saprophyticus | |
| Streptococcus | Streptococcus agalactiae | Gram-positive |
| | Streptococcus pneumoniae | |
| | Streptococcus pyogenes | |
| Treponema | Treponema pallidum | Gram-negative |
| Vibrio | Vibrio cholerae | Gram-negative |
| Yersinia | Yersinia pestis | Gram-negative |
| | Yersinia enterocolitica | |
| | Yersinia pseudotuberculosis | |

The bacterial infection prevented and/or treated by compounds of the present invention may be infection by one or more Gram-positive bacteria. Furthermore, the compounds of the present invention may be selective for one or more Gram-positive bacteria over Gram-negative bacteria. Thus, compounds of the present invention may show no significant inhibition of growth of Gram-negative bacteria.

The bacterial infection prevented and/or treated by compounds of the present invention may be infection by one or more Gram-negative bacteria. Furthermore, the compounds of the present invention may be selective for one or more Gram-negative bacteria over Gram-positive bacteria. Thus, compounds of the present invention may show no significant inhibition of growth of Gram-positive bacteria.

Furthermore, the compounds of the present invention may inhibit the growth of both Gram-positive bacteria and Gram-negative bacteria.

Therapeutic index is the ratio of the dose that produces growth inhibition in 50% of CHO or HEPg2 cells divided by the dose where 50% of S. aureus growth is inhibited. In some embodiments, compounds have a therapeutic index of greater than 1. In other embodiments, compounds have a therapeutic index of greater than 4. In other embodiments, compounds have a therapeutic index of greater than 8.

Representative examples of gram-positive bacteria include Staphylococci (e.g. S. aureus, S. epidermis), Enterococci (e.g. E. faecium, E. faecalis), Clostridia (e.g. C. difficile), Propionibacteria (e.g. P. acnes) and Streptococci.

Bacterial infections in animals are, for example, described in “Pathogenesis of Bacterial Infections in Animals”, edited by Carlton L. Gyles, John F. Prescott, J. Glenn Songer, and Charles O. Thoen, published by Wley-Blackwell (Fourth edition, 2010—ISBN 978-0-8138-1237-3), which is hereby incorporated by reference. Many are the same as listed above for humans.

Combinations

Treatments as described herein may be in combination with one or more know antibiotics, examples of which are described below:

(a) Aminoglyosides: Amikacin, Gentamicin, Kanamycin, Neomycin, Netilmicin, Tobramycin, Paromomycin, Streptomycin; Spectinomycin;

(b) Ansamycins: Geldanamycin, Herbimycin, Rifaximin;

(c) Carbacephem: Loracarbef;

(d) Cabapenems: Ertapenem, Doripenem, Imipenem/Cilastatin, Meropenem;

(e) 1^{st} generation Cephlasporins: Cefadroxil, Cefazolin, Cefalotin or Cefalothin, Cefalexin;

(f) 2^{nd} generation Cephlasporins: Cefaclor, Cefamandole, Cefoxitin, Cefprozil, Cefuroxime;

(g) 3^{rd} generation Cephlasporins: Cefixime, Cefdinir, Cefditoren, Cefoperazone, Cefotaxime, Cefpodoxime, Ceftazidime, Ceftibuten, Ceftizoxime, Ceftriaxone;

(h) 4^{th} generation Cephlasporins: Cefepime;

(i) 5^{th} generation Cephlasporins: Ceftaroline fosamil, Ceftobiprole;

(j) Glycopeptides: Teicoplanin, Vancomycin, Telavancin;

(k) Lincosamides: Clindamycin, Lincomycin

(l) Lipopeptide: Daptomycin

(m) Macrolides: Azithromycin, Clarithromycin, Dirithromycin, Erythromycin, Roxithromycin, Troleandomycin, Telithromycin, Spiramycin;

(n) Monobactams: Aztreonam;

(o) Nitrofurans: Furazolidone, Nitrofurantoin;

(p) Oxazolidonones: Linezolid, Posizolid, Radezolid, Torezolid;

(q) Penicillins: Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin, Ticarcillin;

(r) Polypeptides: Bacitracin, Colistin, Polymyxin B;

(s) Quinolones: Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin;

(t) Sulfonamides: Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, Sulfonamidochrysoidine; and

(u) Tetracylines: Demeclocycline, Doxycycline, Minocycline, Oxytetracycline, Tetracycline.

General Experimental

Compounds of the formula I, where A is S, may be synthesised via the coupling of a chloro phosphine gold (I) complex of formula II with a thiol of formula III:

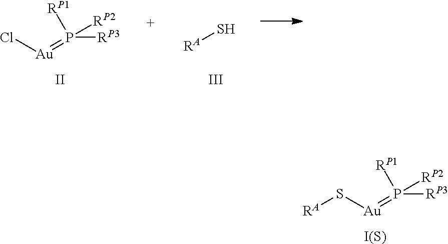

The reaction may take place in an appropriate solvent, such as ethanol, and in the presence of a base, such as K₂CO₃. Heating may be applied, or the reaction may be carried out at room temperature or lower, e.g. 0° C.

Compounds of the formula I, where A is Se, may be synthesised via a two step procedure comprising reduction of a diselenide of formula IV, and then coupling in situ to a chloro phosphine gold (I) complex of formula II:

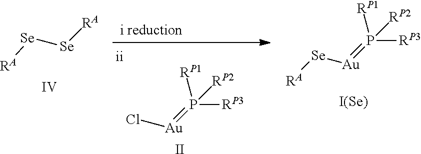

The reduction may take place in an appropriate solvent, such as ethanol, using a reducing agent, such as sodium borohydride. The coupling may take place in the same solvent, and in the presence of a base, such as K₂CO₃. Heating may be applied, or the reaction may be carried out at room temperature or lower, e.g. 0° C.

Isomers, Salts and Solvates

Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

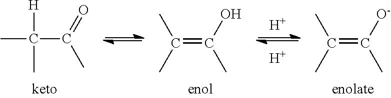

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; Au may be in any isotopic forms, including ¹⁹⁷Au and ¹⁹⁵Au; S may be in any isotopic forms, including ³²S, ³³S, ³⁴S and ³⁶S; P may be in any isotopic forms, including ³¹P, ³³P and ³²P; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Salts

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also include salt forms thereof.

Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

Unless otherwise specified, a reference to a particular compound also include solvate forms thereof.

The Subject/Patient

The subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus. In one preferred embodiment, the subject/patient is a human.

Dosage and Formulation

The dosage administered to a patient will normally be determined by the prescribing physician and will generally vary according to the age, weight and response of the individual patient, as well as the severity of the patient's symptoms and the proposed route of administration. However, in most instances, an effective therapeutic daily dosage will be in the range of from about 0.05 mg/kg to about 100 mg/kg of body weight and, preferably, of from 0.05 mg/kg to about 5 mg/kg of body weight administered in single or divided doses. In some cases, however, it may be necessary to use dosages outside these limits.

While it is possible for an active ingredient to be administered alone as the raw chemical, it is preferable to present it as a pharmaceutical formulation. The formulations, both for veterinary and for human medical use, of the present invention comprise a compound of formula (I) in association with a pharmaceutically acceptable carrier therefor and optionally other therapeutic ingredient(s). The carrier(s) must be ‘acceptable’ in the sense of being compatible with the other ingredients of the formulation and not deleterious to the recipient thereof.

Conveniently, unit doses of a formulation contain between 0.1 mg and 1 g of the active ingredient. Preferably, the formulation is suitable for administration from one to six, such as two to four, times per day. For topical administration, the active ingredient preferably comprises from 1% to 2% by weight of the formulation but the active ingredient may comprise as much as 10% w/w. Formulations suitable for nasal or buccal administration, such as the self-propelling powder-dispensing formulations described hereinafter, may comprise 0.1 to 20% w/w, for example about 2% w/w of active ingredient.

The formulations include those in a form suitable for oral, ophthalmic, rectal, parenteral (including subcutaneous, vaginal, intraperitoneal, intramuscular and intravenous), intra-articular, topical, nasal or buccal administration. The toxicity of certain of the compounds in accordance with the present invention will preclude their administration by systemic routes, and in those, and other, cases opthalmic, topical or buccal administration, and in particular topical administration, is preferred for the treatment of local infection.

Formulations of the present invention suitable for oral administration may be in the form of discrete units such as capsules, cachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. The active ingredient may also be in the form of a bolus, electuary or paste. For such formulations, a range of dilutions of the active ingredient in the vehicle is suitable, such as from 1% to 99%, preferably 5% to 50% and more preferably 10% to 25% dilution.

Formulations for rectal administration may be in the form of a suppository incorporating the active ingredient and a carrier such as cocoa butter, or in the form of an enema.

Formulations suitable for parenteral administration comprise a solution, suspension or emulsion, as described above, conveniently a sterile aqueous preparation of the active ingredient that is preferably isotonic with the blood of the recipient.

Formulations suitable for intra-articular administration may be in the form of a sterile aqueous preparation of the active ingredient, which may be in a microcrystalline form, for example, in the form of an aqueous microcrystalline suspension or as a micellar dispersion or suspension. Liposomal formulations or biodegradable polymer systems may also be used to present the active ingredient particularly for both intra-articular and ophthalmic administration.

Formulations suitable for topical administration include liquid or semi-liquid preparations such as liniments, lotions or applications; oil-in-water or water-in-oil emulsions such as creams, ointments or pastes; or solutions or suspensions such as drops. For example, for ophthalmic administration, the active ingredient may be presented in the form of aqueous eye drops, as for example, a 0.1-1.0% solution.

Drops according to the present invention may comprise sterile aqueous or oily solutions. Preservatives, bactericidal and fungicidal agents suitable for inclusion in the drops are phenylmercuric salts (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the eye. An eye lotion may comprise a sterile aqueous solution optionally containing a bactericide or preservative prepared by methods similar to those for the preparation of drops. Lotions or liniments for application to the skin may also include an agent to hasten drying and to cool the skin, such as an alcohol, or a softener or moisturiser such as glycerol or an oil such as castor oil or arachis oil.

Creams, ointments or pastes according to the present invention are semi-solid formulations of the active ingredient in a base for external application. The base may comprise one or more of a hard, soft or liquid paraffin, glycerol, beeswax, a metallic soap; a mucilage; an oil such as a vegetable oil, eg almond, corn, arachis, castor or olive oil; wool fat or its derivatives; or a fatty acid ester of a fatty acid together with an alcohol such as propylene glycol or macrogols. The formulation may also comprise a suitable surface-active agent, such as an anionic, cationic or non-ionic surfactant such as a glycol or polyoxyethylene derivatives thereof. Suspending agents such as natural gums may be incorporated, optionally with other inorganic materials, such as silicaceous silicas, and other ingredients such as lanolin.

Formulations suitable for administration to the nose or buccal cavity include those suitable for inhalation or insufflation, and include powder, self-propelling and spray formulations such as aerosols and atomisers. The formulations, when dispersed, preferably have a particle size in the range of 10 to 200 μm.

Such formulations may be in the form of a finely comminuted powder for pulmonary administration from a powder inhalation device or self-propelling powder-dispensing formulations, where the active ingredient, as a finely comminuted powder, may comprise up to 99.9% w/w of the formulation.

Self-propelling powder-dispensing formulations preferably comprise dispersed particles of solid active ingredient, and a liquid propellant having a boiling point of below 18° C. at atmospheric pressure. Generally, the propellant constitutes 50 to 99.9% w/w of the formulation whilst the active ingredient constitutes 0.1 to 20% w/w. for example, about 2% w/w, of the formulation.

The pharmaceutically acceptable carrier in such self-propelling formulations may include other constituents in addition to the propellant, in particular a surfactant or a solid diluent or both. Especially valuable are liquid non-ionic surfactants and solid anionic surfactants or mixtures thereof. The liquid non-ionic surfactant may constitute from 0.01 up to 20% w/w of the formulation, though preferably it constitutes below 1% w/w of the formulation. The solid anionic surfactants may constitute from 0.01 up to 20% w/w of the formulation, though preferably below 1% w/w of the composition.

Formulations of the present invention may also be in the form of a self-propelling formulation wherein the active ingredient is present in solution. Such self-propelling formulations may comprise the active ingredient, propellant and co-solvent, and advantageously an antioxidant stabiliser. Suitable co-solvents are lower alkyl alcohols and mixtures thereof. The co-solvent may constitute 5 to 40% w/w of the formulation, though preferably less than 20% w/w of the formulation. Antioxidant stabilisers may be incorporated in such solution-formulations to inhibit deterioration of the active ingredient and are conveniently alkali metal ascorbates or bisulphites. They are preferably present in an amount of up to 0.25% w/w of the formulation.

Formulations of the present invention may also be in the form of an aqueous or dilute alcoholic solution, optionally a sterile solution, of the active ingredient for use in a nebuliser or atomiser, wherein an accelerated air stream is used to produce a fine mist consisting of small droplets of the solution.

In addition to the aforementioned ingredients, the formulations of this invention may include one or more additional ingredients such as diluents, buffers, flavouring agents, binders, surface active agents, thickeners, lubricants, preservatives eg methylhydroxybenzoate (including anti-oxidants), emulsifying agents and the like. A particularly preferred carrier or diluent for use in the formulations of this invention is a lower alkyl ester of a C₁₈ to C₂₄ mono-unsaturated fatty acid, such as oleic acid, for example ethyl oleate. Other suitable carriers or diluents include capric or caprylic esters or triglycerides, or mixtures thereof, such as those caprylic/capric triglycerides sold under the trade name Miglyol, eg Miglyol 810.

Embodiments of the invention will now be described by way of example only.

### EXAMPLES

Analytical Methods

MeCN-FA Method:

Phenomenex Luna C18(2) 3 μm, 4.6×50 mm; H₂O+0.1% formic acid; B=MeCN+0.1% formic acid; 45° C.; 0 min 5%, 1 min 37.5%, 3 min 95%, 3.5 min 95%, 3.51 min 5%, 4.5 min 5%; 2.2-2.3 mL/min.

MeOH-Bicarbonate Method:

Phenomenex Luna C18(2) 3 μm, 4.6×50 mm; H₂O+10 mmol ammonium bicarbonate; B=MeOH; 45° C.; 0 min 5%, 1 min 37.5%, 3 min 95%, 3.5 min 95%, 3.51 min 5%, 4.5 min 5%; 2.2-2.3 mL/min.

Synthesis of Key Intermediates

### 4-Mercapto cyclohexanol (I1)(Used During Synthesis of 6)

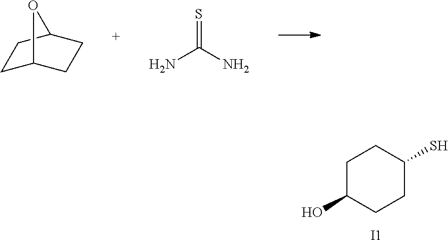

7-Oxabicyclo[2.2.1]heptane (1 mL, 10.2 mmol), p-TsOH (2.6 g, 15.3 mmol) and thiourea (1.2 g, 15.3 mmol) were combined and dissolved in EtOH (10 mL) at rt. The reaction was heated at reflux for 21 h whereupon it was cooled to rt and NaOH (1.3 g, 32.4 mmol) as a solution in H₂O (3 mL) was added in one portion. The resultant suspension dissolved upon heating the reaction mixture at reflux for a further 2 h. The EtOH was removed in vacuo and the aqueous residue cooled to 0° C. before a solution of H₂SO₄ (0.8 mL, 14.5 mmol) in H₂O (5 mL) was added dropwise over the course of 10 minutes. The reaction mixture was diluted with H₂O (20 mL) and extracted with EtOAc (3×40 mL) before passing the organic extracts through a phase separator cartridge. Removal of the solvent under reduced pressure gave the crude product as a yellow oil which was purified by column chromatography (Biotage Isolera 4) eluting with neat EtOAc to provide the title compound as a colourless oil (740 mg, 5.6 mmol, 55%).

### (R)-2-Acetylamino-3-((R)-2-acetylamino-2-methoxycarbonyl-ethyldiselanyl)-propionic acid methyl ester (I2)(Used During Synthesis of 12)

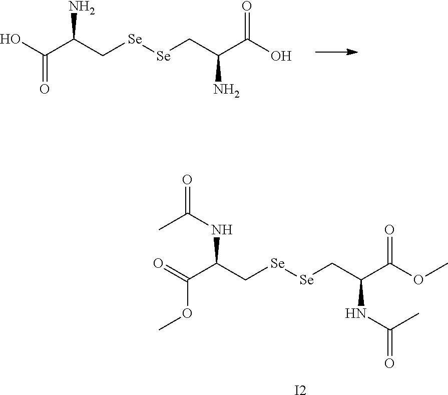 [SMILES: N[C@@H](C[Se][Se]C[C@H](N)C(=O)O)C(=O)O ; COC(=O)[C@H](C[Se][Se]C[C@H](NC(C)=O)C(=O)OC)NC(C)=O ; "I2"]

Anhydrous MeOH (15 mL) was cooled to 0° C. and acetyl chloride (1.6 mL, 22.5 mmol) added dropwise over the course of 5 minutes. The colourless solution was stirred at 0° C. for 10 minutes whereupon L-selenocysteine (500 mg, 1.5 mmol) was added in one portion. The resultant yellow reaction mixture was warmed to rt and stirred at this temperature for 24 h before concentrating in vacuo to give the crude di-selenide ester hydrochloride as a yellow solid. The crude material was re-suspended in DCM (15 mL) and cooled to 0° C. at which point Et₃N (1 mL, 7.5 mmol) was added followed by acetyl chloride (0.3 mL, 4.5 mmol). The reaction was stirred at rt for 4 h, before DCM (30 mL) and H₂O (30 mL) were added. The layers were separated and the aqueous phase extracted with DCM (2×20 mL). The combined organic extracts were passed through a phase separator cartridge and the solvent removed in vacuo to give the crude product as a yellow oil which was purified by column chromatography (Biotage Isolera 4) eluting with neat EtOAc to provide the title compound as a colourless oil (270 mg, 0.6 mmol, 41%).

### Chloro(trialkyl phosphine) gold (I) complexes (I6, I7—Used in the Synthesis of 26 & 27)

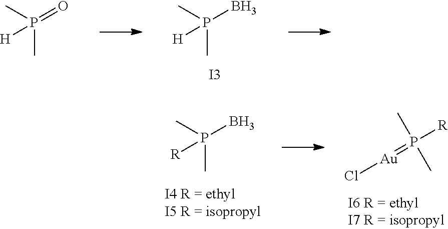

### (a) Dimethylphosphine borane I3

CeCl₃ (25 g, 101.4 mmol) was suspended in THF (100 mL) and stirred at rt for 1 hour. NaBH₄ (3.8 g, 101.4 mmol) was then added and the suspension stirred at rt for a further 1 hour. The reaction was cooled to 0° C. at which point dimethylphosphine oxide (2.6 g, 33.8 mmol) was added drop wise followed by LiAIH₄ (1M in THF, 40.7 mL, 40.7 mmol) also drop wise. The reaction was stirred at rt for 18 h before diluting with toluene (50 mL) then quenching with H₂O (25 mL) and 6N HCl (aq., 25 mL). The suspension was filtered through celite and the layers separated. The aqueous phase was extracted with DCM (3×40 mL) and the combined organic extracts washed with brine (1×40 mL) and passed through a phase separator cartridge. Concentration in vacuo gave the crude product as a yellow oil which was purified by column chromatography (Biotage Isolera 4) eluting with neat iso-hexane to 20% EtOAc/iso-hexane to provide the title compound as a colourless oil (1.49 g, 19.6 mmol, 58%).

### (b) Dimethyl-ethylphosphine borane I4

Dimethylphosphine borane I3 (100 mg, 1.3 mmol) was dissolved in THF (3 mL) and the colourless solution cooled to 0° C. NaH (60% in mineral oil, 53 mg, 1.3 mmol) was added in one portion, whereupon effervescence was observed. The opaque reaction was stirred at rt for 10 minutes then cooled back down to 0° C. whereupon iodoethane (0.12 mL, 1.4 mmol) was added in one portion. When TLC had indicated completion of the reaction, H₂O (10 mL) and Et₂O (10 mL) were added and the phases separated. The aqueous phase was extracted with Et₂O (2×15 mL) and the combined organic extracts washed with brine (1×20 mL) before passing through a phase separator cartridge.

Concentreation in vacuo gave the crude material as a colourless gum. Purification by column chromatography (Biotage Isolera 4) eluting with neat iso-hexane to 20% EtOAc/iso-hexane provided the title compound as a white solid (122 mg, 1.1 mmol, 90%).

### (c) Dimethyl-isopropylphosphine borane I4

Procedure as described for dimethyl-ethylphosphine borane I3, except 2-iodo-2-methyl propane was used instead of iodoethane. The method provided the title compound as a white solid (232 mg, 2.04 mmol, 76%).

### (d) Gold (I) Chloride I6

Dimethyl-ethylphosphine borane I4 (55 mg, 0.53 mmol) was dissolved in THF (5 mL) and the colourless solution degassed with nitrogen for 5 minutes. DABCO (178 mg, 1.6 mmol) was added and the reaction sealed with a Teflon screw cap. The reaction was heated to 100° C. and stirred at this temperature for 4 h before cooling in an ice bath and adding chloro(tetrahydrothiophene)gold(I) (170 mg, 0.53 mmol) in one portion. After stirring at rt for 18 h the reaction was diluted with EtOAc (10 mL) and H₂O (10 mL) and the phases separated. The aqueous phase was extracted with EtOAc (2×20 mL) and the combined organic extracts washed with brine (20 mL) before passing theough a phase separator cartridge. Concentration in vacuo gave the crude product as a brown oil which was purified by column chromatography (Biotage Isolera 4) eluting with neat iso-hexane to 50% EtOAc/iso-hexane to provide the title compound as a colourless oil (16.5 mg, 0.05 mmol, 10%).

### (e) Gold (I) chloride I7

Procedure as described for gold (I) chloride I6, except dimethyl-isopropylphosphine borane I5 was used instead of dimethyl-ethylphosphine borane I4. The method provided the title compound as a white solid (98 mg, 0.29 mmol, 45%).

### Example 1

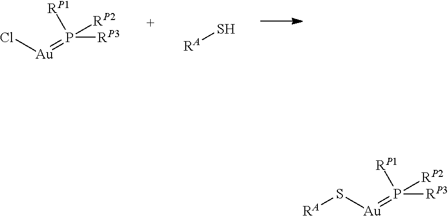

Method A:

To a stirred suspension of the chlorophosphine gold (I) compound (0.32 mmol) in EtOH (1 mL) at 0° C., was slowly added the appropriate thiol (0.32 mmol) as a solution in 10% K₂CO₃ (aq., 1 mL) and EtOH (1 mL). The reaction was stirred at 0° C. for 1 hour before warming to rt and allowing to stir at this temperature for 3 h. Once the reaction had gone to completion (by TLC) the reaction was diluted with H₂O (5 mL) and the solution extracted with DCM (3×15 mL). The combined organic extracts were passed through a phase separator cartridge and the solvent evaporated to provide the title compound.

Method B:

As Method A, except after stirring at 0° C. the reaction was heated at 50° C. for 16 h whereupon a thick white ppt had formed. The solid was collected by filtration, washed with EtOH (1 mL) and H₂O (2 mL) before drying under high vacuum for 24 h to give the title compound.

Method C:

As Method A, except the reaction is stirred at 0° C. for 1 hour only

Method D:

As Method C, except MeOH is used instead of EtOH

The following compounds were made using these methods:

TABLE 2

| | | | |
|---|---|---|---|
| | Compound | | Analytical Data |
| Structure | Number | Method | Yield/Physical appearance |

| | | | |
|---|---|---|---|
| 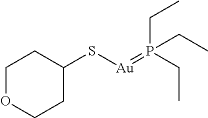 | 1 | A | ¹H-NMR (400 MHz, CDCl₃): δ ppm 1.10 (dt, 9H J = 18.4, 7.8 Hz), 1.64 (m, 2H), 1.74 (dq, 6H, J = 9.6, 7.8 Hz), 1.94 (m, 2H), 3.24-3.36 (m, 3H), 3.85 (m, 2H). ¹³C-NMR (100 MHz, CDCl₃): δ ppm 8.96 (s), 18.06 (d, J = 32.2 Hz), 38.6 (s), 42.34 (s), 68.17 (s). ³¹P- NMR (162 MHz, CDCl₃): δ ppm 37.81 (s). Colourless oil; 115 mg, 83% |
| 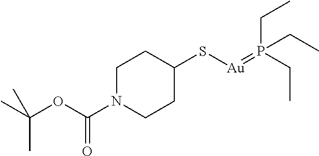 | 2 | A | ¹H-NMR (400 MHz, CDCl₃): δ ppm 1.09 (dt, 9H, J = 18.2, 7.6 Hz), 1.33 (s, 9H), 1.46 (m, 2H), 1.73 (dq, 6H, J = 9.8, 7.6 Hz), 1.95 (m, 2H), 2.69 (m, 2H), 3.26 (m, 1H), 3.92 (2H, br s). ¹³C-NMR (100 MHz, CDCl₃): δ ppm 8.96 (s), 18.06 (d, J = 32.2 Hz), 28.40 (s), 39.67 (s), 40.95 (s), 79.05 (s), 154.80 (s). ³¹P-NMR (162 MHz, CDCl₃): δ ppm 37.74 (s). Yellow oil; 95 mg, 55% |
| 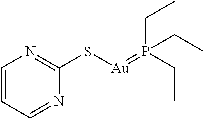 | 3 | A | ¹H-NMR (400 MHz, CDCl₃): δ ppm 1.27 (dt, 9H, J = 18.4, 7.6 Hz), 1.90 (dq, 6H, J = 9.8, 7.6 Hz), 6.82 (t, 1H, J = 4.8 Hz), 8.34 (d, 2H, J = 4.8 Hz). ¹³C-NMR (100 MHz, CDCl₃): δ ppm 9.03 (s), 18.13 (d, J = 33.6 Hz), 115.30 (s), 156.48 (s), 180.86 (s). ³¹P- NMR (162 MHz, CDCl₃): δ ppm 36.72 (s). Colourless oil; 105 mg, 38% |
| 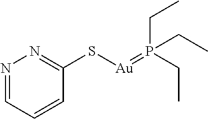 | 4 | A | ¹H-NMR (400 MHz, DMSO-d6): δ ppm 1.16 (dt, 9H, J = 18.7, 7.6 Hz), 1.96 (dq, 6H, J = 10.4, 7.6 Hz), 7.24 (dd, 1H, J = 8.6, 4.6 Hz), 7.50 (dd, 1H, J = 8.6, 1.5 Hz), 8.72 (dd, 1H, J = 4.6, 1.5 Hz). ¹³C-NMR (100 MHz, CDCl₃): δ ppm 9.07 (s), 18.11 (d, J = 33.6 Hz), 125.01 (s), 130.15 (s), 146.84 (s), 172.48 (s). ³¹P-NMR (162 MHz, CDCl₃): δ ppm 36.66 (s). Brown solid; 47 mg, 34% |
| 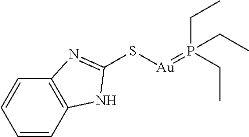 | 5 | B | ¹H-NMR (400 MHz, DMSO-d6): δ ppm 1.16 (dt, 9H, J = 18.7, 7.6 Hz), 1.94 (dq, 6H, J = 10.1, 7.6 Hz), 6.97 (m, 2H), 7.22 (br s, 2H), 11.98 (br s, 1H). ¹³C- NMR (100 MHz, DMSO-d6): δ ppm 8.95 (s), 16.92 (d, J = 34.4 Hz), 120.15 (s). ³¹P-NMR (162 MHz, DMSO-d6): δ ppm 38.08 (s). White solid; 78 mg, 52% |
| 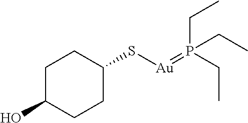 | 6 | A | ¹H-NMR (400 MHz, CDCl3): δ ppm 1.20 (dt, 9H, J = 18.4, 7.6 Hz), 1.33 (m, 2H), 1.52 (m, 2H), 1.84 (dq, 6H, J = 9.8, 7.6 Hz), 1.97 (m, 2H), 2.17 (m, 2H), 3.23 (m, 1H), 3.58 (m, 1H). ¹³C-NMR (100 MHz, DMSO-d6): δ ppm 9.00 (s), 17.09 (d, J = 33.6 Hz), 36.04 (s), 40.67 (s), 42.01 (s), 68.39 (s). ³¹P-NMR (162 MHz, DMSO-d6): δ ppm 38.67 (s). Pale yellow oil; 128 mg, 90% |
| 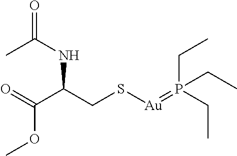 | 7 | C | ¹H-NMR (400 MHz, CDCl3): δ ppm 1.20 (dt, 9H, J = 18.2 Hz, 7.6 Hz), 1.82 (dq, 6H, J = 9.8, 7.6 Hz), 2.05 (s, 3H), 3.32 (dd, 1H, J = 13.1, 4.8 Hz), 3.45 (dd, 1H, J = 13.1, 4.8 Hz), 3.73 (s, 3H), 4.77 (dt, 1H, J = 7.6, 4.8 Hz), 6.66 (d, 1H, J = 7.6 Hz). ¹³C- NMR (100 MHz, CDCl3): δ ppm 8.93 (s), 17.96 (d, J = 32.9 Hz), 23.32 (s), 30.04 (s), 52.29 (s), 54.82 (s), 169.81 (s), 171.66 (s). ³¹P-NMR (162 MHz, CDCl3): δ ppm 36.67 (s). White solid; 72 mg, 45% |
| 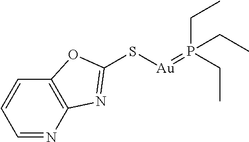 | 8 | A | ¹H-NMR (400 MHz, CDCl3): δ ppm 1.26 (dt, 9H, J = 18.7, 7.6 Hz), 1.92 (dq, 6H, J = 10.1, 7.6 Hz), 7.02 (dd, 1H, J = 8.1, 5.1 Hz), 7.52 (dd, 1H, J = 8.1, 1.5 Hz), 8.32 (dd, 1H, J = 5.1, 1.5 Hz). ¹³C-NMR (100 MHz, CDCl3): δ ppm 9.15 (s), 18.03 (d, J = 34.4 Hz), 99.99 (s), 115.64 (s), 117.58 (s), 144.32 (s), 144.82 (s). ³¹P-NMR (162 MHz, CDCl3): δ ppm 36.44 (s). Pale yellow oil; 136 mg, 91% |
| 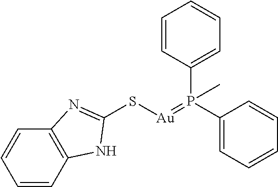 | 9 | B | ¹H-NMR (400 MHz, DMSO-d6): δ ppm 2.30 (d, 3H, J = 10.4 Hz), 6.99 (m, 2H), 7.27 (m, 2H), 7.50-7.60 (m, 6H), 7.82-7.90 (m, 4H), 12.08 (br s. 1H). ¹³C- NMR (100 MHz, DMSO-d6): δ ppm 12.48 (d, J = 36.6 Hz), 120.30 (s), 129.2 (d, J = 11 Hz), 131.44 (d, J = 40.3 Hz), 131.73 (d, J = 13.9 Hz), 132.80 (d, J = 13.9 Hz). ³¹P-NMR (162 MHz, DMSO-d6): δ ppm 24.41 (s). White solid; 142 mg, 89% |
| 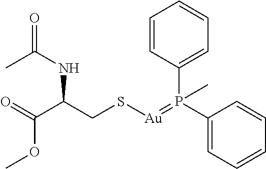 | 10 | C | ¹H-NMR (400 MHz, CDCl3): δ ppm 1.97 (s, 3H), 2.12 (d, 3H, J = 9.6 Hz), 3.39 (dd, 1H, J = 13.4, 4.5 Hz), 3.55 (dd, 1H, J = 13.4, 4.5 Hz), 3.61 (s, 3H), 4.86 (dt, 1H, J = 7.8, 4.5 Hz), 6.73 (d, 1H, J = 7.8 Hz), 7.44-7.54 (m, 6H), 7.60-7.69 (m, 4H). ¹³C- NMR (100 MHz, CDCl3): δ ppm 14.41 (d, J = 35.9 Hz), 23.24 (s), 30.47 (s), 52.27 (s), 54.52 (s), 129.27 (d, J = 10.2 Hz), 131.67 (d, J = 2.2 Hz), 132.64 (d, J = 2.9 Hz), 132.78 (d, J = 2.2 Hz), 169.81 (s), 171.66 (s). ³¹P-NMR (162 MHz, CDCl3): δ ppm 23.30 (s). |
| | | | White solid; 127 mg, 77% |
| 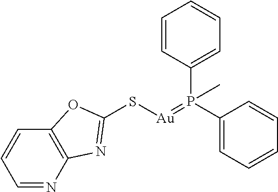 | 11 | A | ¹H-NMR (400 MHz, DMSO-d6): δ ppm 2.41 (d, 3H, J = 10.6 Hz), 7.21 (dd, 1H, J = 8.1, 5.1 Hz), 7.50- 7.65 (m, 6H), 7.80-7.95 (m, 5H), 8.31 (dd, 1H, J = 5.1, 1.5 Hz). ¹³C-NMR (100 MHz, DMSO-d6): δ ppm 12.28 (d, J = 37.3 Hz), 116.35 (s), 118.32 (s), 129.32 (d, J = 11.7 Hz), 130.68 (s), 131.26 (s), 131.85 (d, J = 2.9 Hz), 132.78 (d, J = 13.2 Hz), 143.59 (s), 144.77 (s). ³¹P-NMR (162 MHz, DMSO-d6): δ ppm 24.85 (s). White solid; 108 mg, 60% |
| 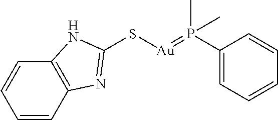 | 13 | A | ¹H-NMR (400 MHz, DMSO-d6) δ ppm 1.92 (d, 6H, J = 10.9 Hz), 6.97-6.99 (m, 2H), 7.24 (br s, 2H), 7.54-7.57 (m, 3H), 7.94-7.99 (m, 2H), 11.98 (br s, 1H). ¹³C-NMR (100 MHz, DMSO-d6): δ ppm 14.46 (d, J = 36.1 Hz), 120.21 (s), 128.92 (d, J = 11.1 Hz), 131.4 (s), 132.04 (d, J = 13.1 Hz), 132.49 (s), 133.06 (s) White solid; 70 mg, 54% |
| 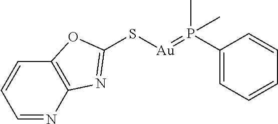 | 14 | A | ¹H-NMR (400 MHz, CDCl₃): δ ppm 1.91 (d, 6H, J = 10.6 Hz), 7.04 (dd, 1H, J = 7.8, 4.8 Hz), 7.52-7.56 (m, 4H), 7.78-7.83 (m, 2H), 8.34 (dd, 1H, J = 4.8, 1.3 Hz). ¹³C-NMR (100 MHz, CDCl₃): δ ppm 15.83 (d, J = 37.2 Hz), 115.75 (s), 117.65 (s), 129.32 (d, J = 12.7 Hz), 131.91 (d, J = 14.1 Hz), 100.45 (s), 144.86 (s), 157.45 (s), 175.48 (s). ³¹P-NMR (162 MHz, CDCl₃): δ ppm |
| | | | 10.06 (s) |
| | | | Orange gum; 65 mg, 50% |
| 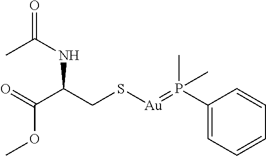 | 15 | A | ¹H-NMR (400 MHz, CDCl₃): δ ppm 1.81 (d, 6H, J = 10.1 Hz), 2.01 (s, 3H), 3.34-3.38 (dd, 1H, J = 13.4, 4.5 Hz), 3.48-3.52 (dd, 1H, J = 13.4, 4.5 Hz), 3.68 (s, 3H), 4.82 (m, 1H), 6.69 (br s, 1H), 7.49-7.53 (m, 3H), 7.70-7.75 (m, 2H). ¹³C-NMR (100 MHz, CDCl₃): δ ppm 15.7 (d, J = 35.2 Hz), 15.77 (d, J = 35.2 Hz), 23.31 (s), 30.40 (s), 52.34 (s), 54.65 (s), 129.24 (d), J = 11.1 Hz), 131.71 (d, J = 8.0 Hz), 131.83 (d, J = 2.0 Hz), 169.82 (s), 171.69 (s). ³¹P- NMR (162 MHz, CDCl_{3):} δ ppm 11.19 (s) |
| | | | White gum; 89 mg, 64% |
| 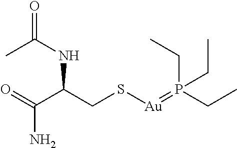 | 16 | A | ¹H-NMR (400 MHz, CDCl₃): δ ppm 1.19 (dt, 9H, J = 18.4, 7.6 Hz), 1.83 (dq, 6H, J = 9.9, 7.6 Hz), 2.03 (s, 3H), 3.09 (dd, 1H, J = 12.9, 7.6 Hz), 3.44 (dd, 1H, J = 12.9, 4.8 Hz), 4.46 (m, 1H), 5.65 (br s, 1H), 6.85 (br s, 1H), 7.06 (br s, 1H) ¹³C-NMR (100 MHz, CDCl₃): δ ppm 9.06 (s), 18.04 (d, J = 33.2 Hz), 23.37 (s), 30.19 (s), 57.05 (s), 170.12 (s), 173.64 (s). ³¹P-NMR (162 MHz, CDCl₃): δ ppm 36.87 (s) White solid; 92 mg, 88% |
| 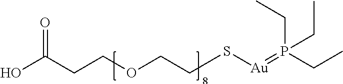 | 17 | A | ¹H-NMR (400 MHz, CDCl₃): δ ppm 1.18 (dt, 9H, J = 18.2, 7.6 Hz), 1.81 (dq, 6H, J = 9.8, 7.6 Hz), 2.60 (t, 2H, J = 6.1 Hz), 3.10-3.14 (m, 2H), 3.61-3.67 (m, 30H), 3.76 (t, 2H, J = 6.1 Hz). ¹³C-NMR (100 MHz, CDCl₃): δ ppm 8.98 (s), 18.06 (d, J = 33.2 Hz), |
| | | | 26.78 (s), 35.14 (s), 66.69 (s), 69.9 9s), 70.23 (s), |
| | | | 70.46 (s), 70.51 (s), 70.55 (s), 70.61 (s), 173.91 (s) |
| | | | ³¹P-NMR (162 MHz, CDCl₃): δ ppm 36.87 (s) |
| | | | Colourless gum; 58 mg, 75% |
| 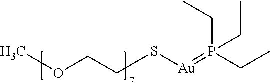 | 18 | A | ¹H-NMR (400 MHz, CDCl₃): δ ppm 1.21 (dt, 9H, J = 18.2, 7.6 Hz), 1.83 (dq, 6H, J = 9.9, 7.6 Hz), 3.13-3.18 (m, 2H), 3.39 (s, 3H), 3.55- 3.58 (m, 2H), 3.65-3.70 (m, 24H). ¹³C-NMR (100 MHz, CDCl₃): δ ppm 8.98 (s), 18.08 (d, J = 32.2 |
| | | | Hz), 26.78 (s), 59.06 (s), 69.92 (s), 70.56 (s), 71.93 |
| | | | (s). ³¹P-NMR (162 MHz, CDCl₃): δ ppm 37.9 (s) |
| | | | Colourless gum; 60 mg, 88% |
| 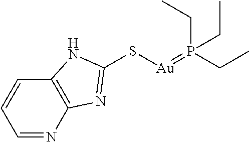 | 19 | C | ¹H-NMR (400 MHz, CDCl3): δ ppm 1.16 (dt, 9H, J = 18.7, 7.6 Hz), 1.97 (m, 6H), 7.00 (dd, 1H, J = 7.3, 5.1 Hz), 7.54 (d, 1H, J = 7.3 Hz), 8.06 (br s, 1H) ³¹P-NMR (162 MHz, CDCl3): δ ppm 39.37 (br s) Brown solid; 28 mg, 18% |
| 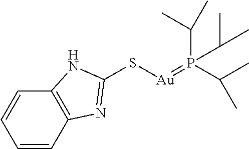 | 20 | B | ¹H-NMR (400 MHz, DMSO-d6): δ ppm 1.29 (dd, 18H, J = 15.9, 7.1 Hz), 2.45 (m, 3H), 6.96 (dd, 2H, J = 5.8, 3.0 Hz), 7.20 (dd, 2H, J = 5.8, 3.0 Hz), 11.91 (br s, 1H). ¹³C-NMR (100 MHz, DMSO-d6): δ ppm 20.18 (d, J = 32.0 Hz), 23.14 (d, J = 29.3 Hz), 120.15 (s). ³¹P-NMR (162 MHz, DMSO-d6): δ ppm 69.68 (s) White solid; 98 mg, 76% |
| 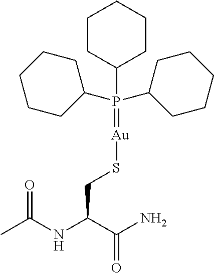 | 24 | C | ¹H-NMR (400 MHz, CDCl₃): δ ppm 1.21-1.35 (m, 9H), 1.39-1.52 (m, 6H), 1.69-1.75 (m, 3H), 1.77-2.01 (m, 15H), 2.03 (s, 3H), 3.11 (dd, 1H, J = 12.9, 7.6 Hz), 3.48 (dd, 1H, J = 12.9, 4.8 Hz), 4.45 (ddd, 1H, J = 7.8, 6.6, 4.8 Hz), 5.42 (br s, 1H), 6.78 (br d, 1H, J = 6.6 Hz), 7.03 (br s, 1H). ³¹P-NMR (162 MHz, CDCl_{3):} δ ppm 57.31 (s) White solid; 60 mg, 93% |
| 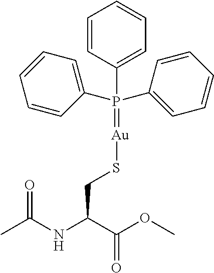 | 25 | C | ¹H-NMR (400 MHz, CDCl₃): δ ppm 1.90 (s, 3H), 3.38 (dd, 1H, J = 13.4, 4.5 Hz), 3.51 (s, 3H), 3.57 (dd, 1H, J = 13.4, 4.0 Hz), 4.86 (dt, 1H, J = 8.1, 4.5 Hz), 6.73 (br d, 1H, J = 8.1 Hz), 7.43-7.56 (m, 15H). ³¹P-NMR (162 MHz, CDCl₃): δ ppm 38.77 (s) White solid; 116 mg, 90% |
| 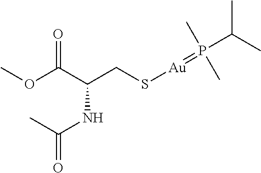 | 26 | D | ¹H NMR (400 MHz, CDCl₃): δ ppm 6.65 (br d, 1H, J = 7.3 Hz), 4.79 (dt, 1H, J = 7.8, 4.8 Hz), 3.75 (s, 3H), 3.45 (dd, 1H, J = 13.1, 4.8 Hz), 3.32 (dd, 1H, J = 13.1, 4.8 Hz), 2.06 (s, 3H), 1.96 (m, 1H), 1.49 (d, 6H, J = 9.9 Hz), 1.25 (d, 3H, J = 6.8 Hz), 1.20 (d, 3H, J = 6.8 Hz). ³¹P-NMR (162 MHz, CDCl₃): δ ppm 22.25 (s). Colourless gum; 52 mg, 84% |
| 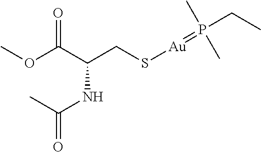 | 27 | D | ¹H NMR (400 MHz, CDCl₃): δ ppm 6.68 (br d, 1H, J = 6.8 Hz), 4.77 (m, 1H), 3.74 (s, 3H), 3.43 (dd, 1H, J = 13.1, 2.3 Hz), 3.31 (dd, 1H, J = 13.1, 2.3 Hz), 2.05 (s, 3H), 1.88-1.78 (m, 2H), 1.51 (d, 6H, J = 10.1 Hz), 1.26-1.15 (m, 3H). ³¹P-NMR (162 MHz, CDCl₃): δ ppm 11.55 (s). Pale yellow gum; 10.5 mg, 54% |
| 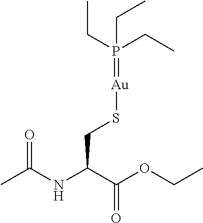 | 28 | C | ¹H-NMR (400 MHz, CDCl₃): δ ppm 1.19 (dt, 9H, J = 18.4, 7.6 Hz), 1.28 (t, 3H, J = 7.3 Hz), 1.82 (dq, 6H, J = 9.9, 7.6 Hz), 2.04 (s, 3H), 3.33 (dd, 1H, J = 13.1, 4.5 Hz), 3.43 (dd, 1H, J = 13.1, 4.8 Hz), 4.19 (dq, 2H, J = 8.6, 7.3 Hz), 4.73 (dt, 1H, J = 7.3, 4.8 Hz), 6.65 (br d, 1H, J = 7.3 Hz). ³¹P-NMR (162 MHz, CDCl₃): ppm 36.51 (s) Colourless gum; 210 mg, 84% |
| 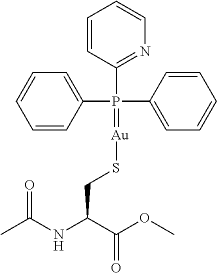 | 29 | C | ¹H-NMR (400 MHz, CDCl₃): δ ppm 1.90 (s, 3H), 3.41 (dd, 1H, J = 13.4, 4.5 Hz), 3.56 (s, 3H), 3.58 (dd, 1H, J = 13.4, 4.0 Hz), 4.89 (dt, 1H, J = 8.0, 4.3 Hz), 6.83 (br d, 1H, J = 8.0 Hz), 7.34-7.39 (m, 1H), 7.43-7.51 (m, 6H), 7.61-7.69 (m, 4H), 7.73-7.82 (m, 2H), 8.76-8.80 (m, 1H). ³¹P- NMR (162 MHz, CDCl₃): δ ppm 34.57 (s) White solid; 104 mg, 81% |

### Example 2

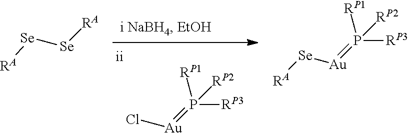

The appropriated diselenide (0.23 mmol) was dissolved in EtOH (4 mL) and the reaction cooled to 0° C. NaBH₄ (17 mg, 0.46 mmol) was added in one portion and the pale yellow solution stirred at 0° C. for 20 min. The chlorophosphine gold (I) compound (0.46 mmol) was then added in one portion and the reaction warmed to rt and stirred at this temperature for 3 hour. The reaction mixture was diluted with DCM (30 mL) and subsequently washed with saturated NH₄Cl (aq., 20 mL), saturated NaHCO₃ (aq., 20 mL) and finally water (20 mL). The organic phase was passed through a phase separator cartridge and the solvent removed in vacuo to give a brown oil which was purified by column chromatography (Biotage Isolera 4) eluting with neat EtOAc to 1:1 EtOAc-WIPE 129 to provide the title compound.

The following compounds were made using these methods:

TABLE 2

| | Compound | Analytical Data |
|---|---|---|
| Structure | Number | Yield/Physical appearance |
| 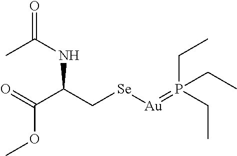 | 12 | ¹H-NMR (400 MHz, CDCl3): δ ppm 1.21 (dt, 9H, J = 18.2, 7.6 Hz), 1.83 (dq, 6H, J = 9.8, 7.6 Hz), 2.05 (s, 3H), 3.24 (dd, 1H, J = 12.1, 4.8 Hz), 3. 34 (dd, 1H, J = 12.1, 4.8 Hz), 3.74 (s, 3H), 4.81 (dt, 1H, J = 7.6, 4.8 Hz), 6.57 (d, 1H, J = 7.6 Hz). ¹³C-NMR (100 MHz, DMSO-d6): δ ppm 8.81 (s), 15.67 (s), 17.07 (d, J = 32.2 Hz), 22.35 (s), 51.66 (s), 56.88 (s), 169.06 (s), 171.66 (s). ³¹P-NMR (162 MHz, DMSO-d6): δ ppm 39.62 (s). White solid; 191 mg, 67% |
| 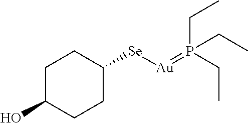 | 21 | ¹H-NMR (400 MHz, CDCl3): δ ppm 1.20 (dt, 9H, J = 18.2, 7.6 Hz), 1.28 (m, 2H), 1.66 (m, 2H), 1.82 (dq, 6H, J = 9.6, 7.6 Hz), 1.92 (m, 2H), 2.24 (m, 2H), 3.36 (m, 1H), 3.58 (m, 1H). ¹³C-NMR (100 MHz, CDCl3): δ ppm 8.90 (s), 17.29 (d, J = 32.2 Hz), 32.69 (s), 36.98 (s), 40.67 (s), 68.27 (s). ³¹P-NMR (162 MHz, DMSO-d6): δ ppm 40.33 (s) Colourless gum; 48 mg, 87% |
| 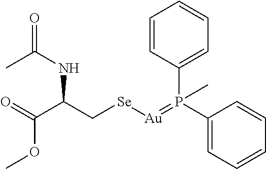 | 22 | ¹H-NMR (400 MHz, DMSO-d6): δ ppm 1.79 (s, 3H), 2.24 (d, 3H, J = 9.9 Hz), 2.95 (dd, 1H, J = 11.9, 7.3 Hz), 3.08 (dd, 1H, J = 11.9, 6.0 Hz), 3.58 (s, 3H), 4.46 (dt, 1H, J = 7.6, 6.0 Hz), 7.49-7.58 (m, 6H), 7.72-7.80 (m, 4H), 8.19 (d, 1H, J = 7.6 Hz). ¹³C-NMR (100 MHz, DMSO-d6): δ ppm 12.47 (d, J = 35.1 Hz), 16.26 (s), 22.33 (s), 51.70 (s), 56.83 (s), 129.15 (d, J = 11.7 Hz), 131.43 (d, J = 2.9 Hz), 132.00 (d, J = 54.2 Hz), 132.55 (d, J = 13.9 Hz), 169.12 (s), 171.67 (s). ³¹P-NMR (162 MHz, DMSO-d6): δ ppm 25.21 (s) Clear gum; 47 mg, 85% |
| 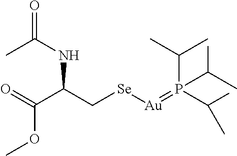 | 23 | ¹H-NMR (400 MHz, DMSO-d6): δ ppm 1.25 (dd, 18H, J = 15.9, 7.1 Hz), 1.83 (s, 3H), 2.40 (m, 3H), 2.84 (dd, 1H, J = 11.6, 7.6 Hz), 3. 01 (dd, 1H, J = 11.6, 6.3 Hz), 3.60 (s, 3H), 4.39 (dt, 1H, J = 7.6, 6.3 Hz), 8.14 (d, 1H, J = 7.6 Hz). ¹³C-NMR (100 MHz, DMSO-d6): δ ppm 15.56 (s), 19.94 (s), 22.32 (s), 23.17 (d, J = 27.1 Hz), 51.59 (s), 57.16 (s), 168.95 (s), 171.69 (s). ³¹P-NMR (162 MHz, DMSO-d6): δ ppm 68.95 (s) White solid; 23 mg, 44% |

### Example 3

Growth Media

Tryptic Soy Broth

| Formula/Litre | |
|---|---|
| Pancreatic Digest of Casein | 17.0 g |
| Enzymatic Digest of Soybean | 3.0 g |
| Sodium Chloride | 5.0 g |
| Di-potassium hydrogen Phosphate | 2.5 g |
| Glucose | 2.5 g |

Directions for use: Dissolve 30 g of the medium in one litre of purified water, mix thoroughly, and then autoclave at 121° C. for 15 minutes.

Luria Broth

| Formula/Litre | |
|---|---|
| Tryptone | 10.0 g |
| Yeast Extract | 5.0 g |
| NaCl | 5.0 g |

Directions for use: Dissolve components in 1 litre of distilled or deionized water and sterilize by autoclaving at 121° C. for 15 minutes.

Mueller Hinton II Broth (Cation-Adiusted)

| Formula/Litre | |
|---|---|
| Beef Extract | 3.0 g |
| Acid Hydrolysate of Casein | 17.5 g |
| Starch | 1.5 g |

*Adjusted and/or supplemented as required with appropriate salts to provide 20-25 mg/L of calcium and 10-12.5 mg/L of magnesium and as additionally required to meet performance criteria.

Directions for use: Dissolve components in 1 litre of distilled or deionized water andand sterilize by autoclaving at 121° C. for 15 minutes.

Brain Heart Infusion Broth

| Formula/Litre | |
|---|---|
| Brain Heart Infusion solids | 12.5 g |
| Beef heart infusion solids | 5 g |
| Proteose peptone | 10 g |
| Glucose | 2 g |
| Sodium Chloride | 5 g |
| Di-sodium Phosphate | 2.5 g |

Directions for use: Dissolve components in 1 litre of purified water. Heat the mixture with frequent agitation to completely dissolve the medium, and sterilize by autoclaving at 121° C. for 15 minutes.

Growth Assay for S. aureus. (NCTC8325)

Stock solution of the test compounds (20 mg/ml) in dimethyl sulfoxide (DMSO) were serially diluted in DMSO and each diluted compound added in duplicate to a 96-well plate to a final DMSO concentration of 2% (v/v). An overnight culture of S. aureus (Oxford strain) grown in tryptic soy broth (TSB) was diluted to approximately 5×10⁷ cfu/ml and 150 μl of this sample was added to each well of the 96-well plates. Control wells included an ‘untreated’ control with bacteria in TSB in the presence of 2% DMSO and a negative sample (containing 150 μl TSB growth media in the presence of 2% DMSO). Plates were incubated in a shaking incubator at 37° C. for 22 hours and bacterial growth assessed by absorbance at a wavelength of 595 nm. The minimum inhibitory concentration (MIC) was defined as the lowest concentration of compound that inhibited growth compared to the no-treatment control.

Variation of Growth Assays for:

Klebsiella pneumoniae (NCTC 13443), Vibrio cholerae or E. coli (ATCC 25922): use of 1/100 overnight dilution to set up assay, medium used: Luria broth (LB); incubation without shaking.

P. aeruginosa (ATCC 27853): use of 1/100 overnight dilution to set up assay, medium used: Cation adjusted Mueller Hinton broth (CaMHB); incubation without shaking.

Enterococcus feacalis (ATCC29212): use of 1/100 overnight dilution to set up assay, medium used: brain heart infusion broth containing 0.5% yeast extract; incubation without shaking.

| | S. aureus | E. faecalis | K. pneumoniae | E. coli | P. aeruginosa | V. cholerae |
|---|---|---|---|---|---|---|
| | MIC | MIC | MIC | MIC | MIC | MIC |
| Compound | (μg/mL) | (μg/mL) | (μg/mL) | (μg/mL) | (μg/mL) | (μg/mL) |
| 1 | 0.8 | | 50 | | | 1.3 |
| 2 | 0.8 | 1.6 | >100 | 50 | 100 | 2.5 |
| 3 | 0.4-0.8 | 1.6 | 6.3-12 | 12.5 | 12.5-25 | 1.3 |
| 4 | 1.6 | 3.1 | 12.5 | 12.5-25 | 25 | 1.6 |
| 5 | 1.6 | 1.6 | 12.5 | 6.3-12.5 | 50 | 1.6 |
| 6 | 0.8 | 1.6 | 50 | 12.5 | 25-50 | 1.6 |
| 7 | 1.3-1.6 | 1.6 | 100 | 25 | 50 | 1.6-3.1 |
| 8 | 1.3-1.6 | 1.6 | 25-50 | 12.5 | 25-50 | 1.56 |
| 9 | 3.1-6.3 | 6.3-12.5 | >100 | >100 | >100 | 3.1-6.3 |
| 10 | 3.1 | 6.3-12.5 | 50-100 | 50-100 | 50 | 3.1-6.3 |
| 11 | 3.1-6.3 | 12.5 | >100 | >100 | >100 | 3.1-6.3 |
| 12 | 1.5-3 | 3.1 | >100 | >100 | >100 | >100 |
| 13 | 3.1 | | 25 | 25 | | |
| 14 | 3.1 | | 25 | 50 | | |
| 15 | 3.1 | | 50 | 100 | | |
| 16 | 0.8 | | 100 | 25-50 | | |
| 17 | 1.6 | | >100 | 100 | | |
| 18 | 1.6 | | >100 | 50 | | |
| 19 | <0.8 | | 12.5 | 12.5 | | |
| 20 | 1.6 | | 50-100 | 25 | | |
| 21 | 0.8 | | >100 | >100 | | |
| 22 | 3.1-6.3 | | >100 | >100 | | |
| 23 | 3.1-6.3 | | >100 | >100 | | |
| 24 | 12.5 | | >100 | >100 | >100 | 12.5 |
| 25 | 6.3 | | >100 | >100 | >100 | 12.5 |
| 26 | 1.6 | | 25 | 12.5-25 | 50 | |
| 27 | 0.8-1.6 | | 6.3 | 6.3 | 12.5-25 | |
| 28 | 0.8 | | 50-100 | | 50-100 | |
| 29 | 3.1 | | >100 | | 100 | |

CHO Toxicity Assay

Cell counting kit-8 (Sigma, CCK-8) assays were performed to assess the effect of compounds on cell viability. The assay is based on the reduction of a water-soluble tetrazolium salt (WST-8) by cellular dehydrogenases to a formazan dye which can be detected spectroscopically. 96-well plates were seeded with chinese hamster ovary cells (CHO) cells at 7×10³ cells per well in Dulbecco's modified Eagle's medium nutrient mixture F-12 Ham (containing 15 mM HEPES, NaHCO₃, pyridoxine and L-glutamine) supplemented with 10% fetal bovine serum (FBS). The following day serial dilutions of compounds (dissolved and diluted in DMSO) were added to the cells in duplicates. Control included an ‘untreated’ control where cells were grown in the presence of 1% DMSO and a medium only control (plus 1% DMSO). After 24 hours CCK-8 reagent (10 μl) was added to each well and cell viability was assessed by measuring the absorbance at a wavelength of 450 nm after 2.5-3 hours. Only living cells can reduce the tetrazolium salts into coloured formazan products. Results were expressed as 50% growth inhibition (TD₅₀) values compared to ‘untreated’ control.

The therapeutic index was calculated as the ratio of the dose that produces growth inhibition in 50% of CHO cells divided by the dose where 50% of S. aureus growth is inhibited.

| | CHO cell | |
|---|---|---|
| Compound | TD ₅₀ (μg/mL) | Therapeutic Index |
| 1 | 0.5 | 1.7 |
| 2 | 0.7 | 2.3 |
| 3 | 0.6 | 3 |
| 4 | 1.4 | 3.7 |
| 5 | 1.5 | 3.5 |
| 6 | 1.5 | 4.5 |
| 7 | 2.6 | 8.7 |
| 8 | 1.6 | 4 |
| 9 | 2.6 | 1.6 |
| 10 | 1.2 | 1.1 |
| 11 | 2.0 | 1.3 |
| 12 | 6.5 | 5.3 |

HepG2 Cell Inhibition Assay

Cell counting kit-8 (Sigma, CCK-8) assays were performed to assess the effect of compounds on cell viability. The assay is based on the reduction of a water-soluble tetrazolium salt (WST-8) by cellular dehydrogenases to a formazan dye which can be detected spectroscopically. 96-well plates were seeded with the human hepatocyte cell line (HepG2) at approximately 8×10³ cells per well in Minimum Essential Medium Eagle (EMEM) with Earle's salts and sodium bicarbonate supplemented with 10% heat-inactivated foetal bovine serum 2 mM glutamine and 1% non-essential amino acids (NEAA). The following day serial dilutions of compounds (dissolved and diluted in DMSO) were added to the cells in duplicates. Control included an ‘untreated’ control where cells were grown in the presence of 1% DMSO and a medium only control (plus 1% DMSO). After 24 hours CCK-8 reagent (10 μl) was added to each well and cell viability was assessed by measuring the absorbance at a wavelength of 450 nm after 2-3 h hours. Only living cells can reduce the tetrazolium salts into coloured formazan products. Results were expressed as 50% growth inhibition (TD₅₀) values compared to ‘untreated’ control. The therapeutic index was calculated as the ratio of the dose that produces growth inhibition in 50% of HepG2 cells divided by the dose where 50% of S. aureus growth is inhibited.

| | HepG2 cell | Therapeutic Index |
|---|---|---|
| Compound | TD₅₀ (μg/mL) | (HepG2) |
| 13 | 8.5 | 6 |
| 14 | 9 | 6 |
| 15 | 6 | 4 |
| 16 | 9 | >11 |
| 17 | 18 | >22 |
| 18 | 9 | >11 |
| 20 | 6 | >8 |
| 22 | 10 | 4 |
| 23 | 12 | 13 |

Efficacy Studies in the Galleria mellonella Model

G. mellonella larvae at 5th or 6th instar stage were purchased from a commercial supplier and used within 3 days. Prior to infection larvae were kept at room temperature. Larvae were infected with bacteria (various Gram positive and negative bacteria, including S. aureus, K. pneumoniae, E. coli and P. aeruginosa) using a sterile Hamilton syringe. Bacteria cultures were grown overnight, washed ×3 in PBS and resuspended in PBS. Larvae were wiped with 70% ethanol and 10 μl of bacteria solution (to cause 80% death within 3-4 days) was injected into the bottom right proleg of the larvae. Larvae injected with 10 μl of PBS were used as negative controls. Larvae were then placed in petri dishes (1 dish per condition) containing filter paper at the bottom of the dish at 37° C. After various time points post infection (1-6 h), larvae were taken from the incubator wiped again with 70% ethanol and injected with 10 μl of various concentrations of compound, dissolved in either 5% dimethyl sulfoxide, 5% ethanol or 5% 1-methyl-2-pyrrolidinone into a proleg on the left hand-side. Control larvae received 10 μl of 5% solvent. Ten larvae were injected for each condition. To assess the toxicity of the compound, larvae were injected with various concentrations of compound alone. Larvae were returned to a 37° C. incubator and checked daily. Larvae were considered dead when no movement occurred when touched with a blunt pair of forceps. Black or discoloured larvae which still showed movement were considered to be alive. Numbers of dead larvae were recorded each day.

Biofilm Prevention Assay

The effect of a test compound on the formation of a S. aureus biofilm may be assessed using a biofilm prevention assay as described by Merritt et al. Current Protocols in Microbiology, 2011, 1B.1.1-1B1.18 with slight modifications. Briefly, S. aureus NCTC 8325, MRSA (RPAH18) and MRSA (MW2) are grown overnight in Tryptic soy broth (TSB) and diluted to between 1/50 and 1/100 before 150 μL is added to the wells of a flat bottomed 96-well plate. Three microliters of auranofin at the appropriate dilution in DMSO are added to the wells in duplicate. Controls included a serial dilution of lincomycin in ethanol (to assess plate to plate variation), a positive control with bacteria alone in TSB with 2% DMSO and a negative (no bacteria) control with 150 μL TSB containing 2% DMSO. Plates are sealed with AeraSeal™ and incubated at 37° C. for 24 hours. The plates are then washed three times with PBS, dried at 60° C. for 1 hour and stained with crystal violet for 1 hour. The plates are again washed three times with water, dried and scanned prior to the addition of 33% acetic acid to re-solubilize the crystal violet stain bound to the adherent cells. Absorbance is then measured at 595 nm and expressed as a percentage of the bacteria only control.

The effect of a test compound on preformed S. aureus biofilms can also be assessed. Briefly S. aureus NCTC 8325 is plated in 96-well plates as described in above and incubated 37° C. for 24 hours. Biofilms are then washed 3 times with TSB and 150 μL of fresh TSB and 3 μL of auranofin at the appropriate dilution in DMSO was added to the wells in duplicate. Plates are again sealed with AeraSeal™ and reincubated 37° C. for 24 hours. Biofilm is then detected as described above.

Persister Cell Assay

To determine whether S. aureus persister cells are susceptible to treatment with a test compound, a persister cell (or SCV) isolate hemB mutant of NCTC 8325-4 may be used (Von Eiff et al., (1997) J Bacteriol 179:4706-4712). This persister cell variant displays varying resistance to erythromycin and the aminoglycosides gentamicin and kanamycin. Growth assays are performed essentially as described above with the bacteria being grown in TSB. Disc assays were also performed by plating bacteria on TSB agar. Discs impregnated with an amount of test compound were placed on top of the agar. The plates were incubated overnight at 37° C. and any zone of bacterial inhibition was observed.

### Abbreviations

aq. Aqueous

br Broad

d Doublet

DCM Dichloromethane

DMSO Dimethyl sulfoxide

Et Ethyl

EtOAc Ethyl acetate

EtOH Ethanol

Et₂O Diethyl ether

FA Formic acid

g Gram

h Hours

ⁱPr Isopropyl

J Coupling constant

LC-MS Liquid chromatography-mass spectrometry

Me Methyl

MeCN Acetonitrile

MeOH Methanol

mg Milligram

min Minutes

mL Millilitre

mmol Millimole

ppm Parts per million

ppt Precipitate

q Quartet

rt Room temperature

s Singlet

TLC Thin layer chromatography

t Triplet

WIPE Water/isopropanol/Ethyl acetate (1:2:9)

### REFERENCES

| | doi |
|---|---|
| Gli\check{s}ić, BD & Djuran MI, Dalton Trans., 2014, | 10.1039/c4dt00022f |
| 43, 5950-5969 | |
| Medeira, JM, et al., Inflammopharmacology, | 10.1007/s10787-012- |
| 2012, 20(6), 297-306 | 0149-1 |
| Jackson-Rosario, S, et al., J. Biol. Inorg. Chem., | 10.1007/s00775-009- |
| 2009, 14(4), 507-519 | 0466-z |
| Novelli, F, et al., Farmaco, 1999, 54, 232-236 | 10.1016/S0014- |
| | 827X(99)00019-1 |
| Shaw, CF, Chem Rev., 1999, 99(9), 2589-2600 | 10.1021/cr980431o |
| Rhodes, MD, et al., J. Inorg. Biochem., 1992, | 10.1016/0162- |
| 46, 129-142 | 0134(92)80016-O |
| Fricker, SP, Transition Met. Chem., 1996, | 10.1007/BF00139037 |
| 21, 377-383 | |

1. A compound of formula (I): 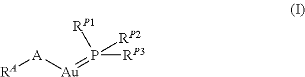
for use in the prevention or treatment of a bacterial infection wherein:
R^{P1} is either methyl, ethyl, isopropyl, cyclohexyl or phenyl;
R^{P2} is selected from methyl, ethyl, isopropyl, cyclohexyl and phenyl;
R^{P3} is either ethyl, isopropyl, cyclohexyl, phenyl or pyridyl;
A is S;
R^{A} is selected from: 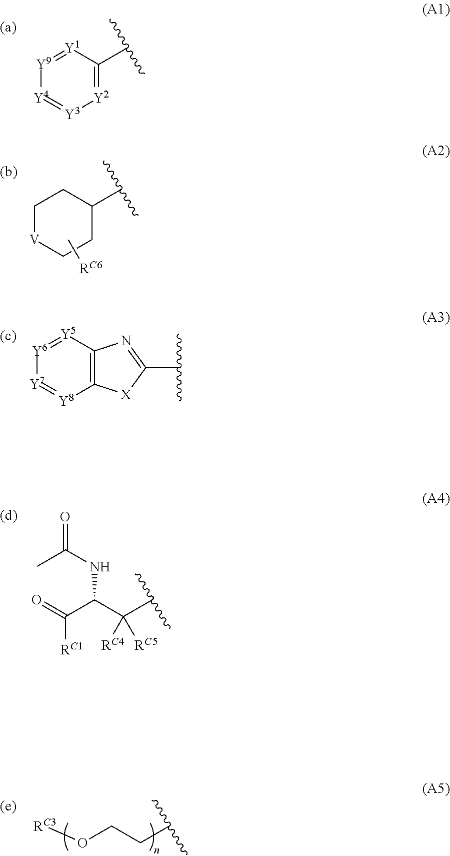
wherein:
each of Y¹, Y², Y³, Y⁴ and Y⁹ is independently selected from CH or N, wherein at least three of Y¹, Y², Y³, Y⁴ and Y⁹ are CH;
V is selected from O, CH—OR^{O1}, N—CO₂—R^{C2} or N—R^{N2};
one of Y⁵, Y⁶, Y⁷ and Y⁸ is selected from CH and N, and the others are CH;
X is selected from NH, S or O;
R^{C1} is selected from O—R^{O2} or NHR^{N1};
R^{O1} is selected from H and C₁₋₃ unbranched alkyl;
R^{O2} is C₁₋₃ unbranched alkyl;
R^{N1} is selected from H and C₁₋₃ unbranched alkyl;
R^{N2} is C₁₋₃ unbranched alkyl;
R^{C2} is either C₁₋₃ unbranched alkyl or C₃₋₄ branched alkyl;
R^{C3} is selected from C₁₋₃ unbranched alkyl and C₂H₄CO₂H;
R^{C4} is either H or Me;
R^{C5} is either H or Me;
R^{C6} represents one or two optional methyl substituents; and
n is an integer from 2 to 8.

2. A compound according to claim 1, wherein (a) R^{P1} and R^{P3} are the same or (b) R^{P1} and R^{P2} are the same.

3. A compound according to claim 2, wherein:
(a) R^{P1}, R^{P2} and R^{P3} are ethyl; or
(b) R^{P1}, R^{P2} and R^{P3} are isopropyl; or
(c) R^{P1} and R^{P3} are phenyl and R^{P2} is methyl; or
(d) R^{P1} and R^{P2} are methyl and R^{P3} is phenyl.

4-6. (canceled)

7. A compound according to claim 1,
wherein R^{A} is A1: 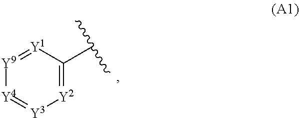
and either:
(a) one of Y¹, Y², Y³, Y⁴ and Y⁹ is N; or
(b) two of Y¹, Y², Y³, Y⁴ and Y⁹ are N; or
(c) R^{A} is phenyl.

8-10. (canceled)

11. A compound according to claim 1,
wherein R^{A} is A2: 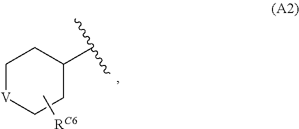
and either:
(a) V is O, or
(b) V is CH—OR^{O1}; or
(c) V is N—CO₂—R^{C2}; or
(d) V is N—R^{N2}.

12-18. (canceled)

19. A compound according to claim 11, wherein there are no optional methyl substituents.

20. A compound according to claim 11, wherein there is a single methyl substituent represented by R^{C6}.

21. A compound according to claim 11, wherein there are two methyl substituents represented by R^{C6}.

22. A compound according to claim 1, wherein R^{A} is A3: 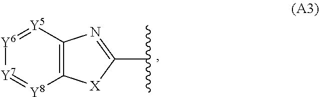
and either:
(a) X is O and one of Y⁵, Y⁶, Y⁷ and Y⁸ is N; or
(b) X is NH and Y⁵, Y⁶, Y⁷ and Y⁸ is are CH.

23-24. (canceled)

25. A compound according to claim 1,
wherein R^{A} is R^{A} is A4: 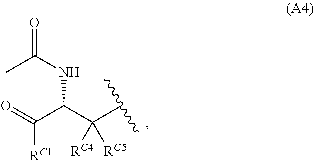
and either:
(a) R^{C1} is O—R^{O2} where R^{O2} is methyl; or
(b) R^{C1} is NHR^{N1}, and R^{N1} is H.

26-27. (canceled)

28. A compound according to claim 25, wherein either:
(a) R^{C4} and R^{C5} are both H; or
(b) R^{C4} is H and R^{C5} is Me; or
(c) R^{C4} and R^{C5} are both Me.

29-30. (canceled)

31. A compound according to claim 1,
wherein R^{A} is A5: 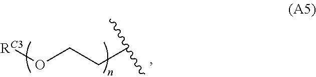
and either:
(a) R^{C3} is methyl; or
(b) R^{C3} is C₂H₄CO₂H.

32-33. (canceled)

34. A compound according to claim 31, wherein n is an integer from 4 to 8.

35. A compound according to claim 1, wherein the bacterial infection prevented and/or treated is infection by one or more Gram-positive bacteria.

36. A compound according to claim 1, wherein the bacterial infection prevented and/or treated is infection by one or more Gram-negative bacteria.

37-44. (canceled)

45. A method of removing or eliminating an existing biofilm, inhibiting biofilmformation, reducing the biomass of a biofilm, promoting the dispersal of microorganisms from a biofilm, sensitizing a microorganism in a biofilm to an antimicrobial agent, killing a microorganism within a biofilm, treating or preventing an infection, disease or disorder caused by a biofilm, inhibiting the growth of a microbial persister cell, killing a microbial persister cell, or treating or preventing an infection, disease or disorder caused by or associated with a microbial persister cell; the method comprising exposing the biofilm to an effective amount of a compound as described in claim 1.

46-47. (canceled)

48. The method according to claim 45, wherein the biofilm comprises bacteria, or the microbial persister cells are bacteria.

49-52. (canceled)

53. The method according to claim 45, comprising further administering at least one additional antimicrobial agent.

54. A medical device coated or impregnated with a compound as described in claim 1.

55. A compound of formula (I): 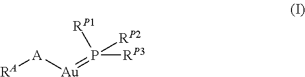
wherein R^{P}% R^{P2}, R^{P3}, A and R^{A} are as defined in claim 1, with the proviso that when R^{A} is (A1), then Y¹, Y² and Y⁹ are CH and Y³ and Y⁴ are N; and with the further proviso that when R^{A} is A3, then one of Y⁵, Y⁶, Y⁷ and Y⁸ is N.

56. A pharmaceutical composition comprising a compound according to claim 55.

57. A pharmaceutical composition according to claim 56, which also comprises a pharmaceutical acceptable diluent or excipient.

58. (canceled)
